(19) United States
(12) Patent Application Publication
(10) Pub. No.: US 2024/0009191 A1
(43) Pub. Date: Jan. 11, 2024

(54) METHOD AND COMPOSITION FOR THE TREATMENT OF DISEASE

(71) Applicant: DIMERIX BIOSCIENCE PTY LTD, Fitzroy, Victoria (AU)

(72) Inventors: Robert Pearson Shepherd, Upwey, Victoria (AU); Kevin Donald George Pfleger, Nedlands, Western Australia (AU)

(21) Appl. No.: 17/675,320

(22) PCT Filed: Sep. 18, 2020
(86) PCT No.: PCT/AU2020/050987

§ 371 Date: Feb. 18, 2022

(30) Foreign Application Priority Data
Sep. 26, 2019 (AU) 2019903606

(51) Int. Cl. A61K 31/506; A61K 31/4192; A61K 31/17; A61K 31/341; A61K 31/4462; A61K 31/4184; A61K 31/4245; A61K 31/4178; A61K 31/41; A61P 11/00

(52) U.S. Cl. CPC A61K 31/506; A61K 31/4192; A61K 31/17; A61K 31/341; A61K 31/4462; A61K 31/4184; A61K 31/4245; A61K 31/4178; A61K 31/41; A61P 11/00

(57) ABSTRACT

A pharmaceutical formulation comprising at least one angiotensin type 1 receptor (AT1R) blocker and at least one CXC chemokine receptor 2 (CXCR2) pathway inhibitor.

### TECHNICAL FIELD

The present invention relates to methods and formulations for the treatment and prevention of chronic obstructive pulmonary disease, comprising (a) at least one angiotensin receptor inhibitor, and (b) at least one C-X-C Motif Chemokine Receptor 2 inhibitor.

### BACKGROUND ART

Chronic Obstructive Pulmonary Disease (COPD) is an umbrella term used to describe progressive lung diseases including emphysema, chronic bronchitis, bronchiectasis and refractory (non-reversible) asthma. These diseases are characterized by increasing breathlessness due to airflow limitations in the lungs. The primary cause of COPD is exposure to tobacco smoke (either active smoking or secondary smoke), however other risk factors include exposure to indoor and outdoor air pollution and occupational dusts and fumes.

COPD is the third-leading cause of death in the world and, though treatments exist to improve the symptoms of COPD, there is currently no targeted treatment registered to slow progression of the condition or cure it. Moreover, among the top five causes of death, this disease is the only one with increasing mortality rates. There is a significant unmet need in COPD, which is recognised by key organisations such as the NIH and globally by the WHO and the CDC. In 2017, the NIH released the COPD National Action Plan in an effort to support research, diagnosis and treatment of the disease. Following this recognition, in 2018 the FDA issued a Guidance to Industry to help sponsors developing drugs to treat COPD. The new guidance will facilitate shorter clinical trials using surrogate and subject-reported endpoints.

COPD is characterised by a limitation of pulmonary airflow that is not fully reversible and is usually progressive with an abnormal inflammatory response (Rabe et al., 2007). No targeted medication exists that prevents the long-term decline in lung function of COPD subjects. However, inhaled anticholinergics, β-adrenergic bronchodilators, and corticosteroids are used to treat the symptoms and exacerbations of COPD. New products that include fixed dose combinations of these drug classes are being approved at a consistent rate, and there are late stage trials of compounds with new mechanisms of actions in progress.

In emphysema the alveoli are damaged; over time, the inner walls of the alveoli weaken and rupture—creating larger air spaces instead of many small ones. This reduces the surface area of the lungs and, in turn, the amount of oxygen that reaches the bloodstream.

Bronchitis is an inflammation of the lining of the bronchial tubes. Bronchiectasis is characterised by abnormal, irreversible bronchial dilatation or a fixed increase in airway diameter. Both are characterized by daily cough and mucus (sputum) production.

Refractory (non-reversible) asthma does not respond to usual asthma medications. In an asthma attack, bronchial airways tighten up and swell. Medications can usually reverse this, opening up the airways and returning them to their pre-attack state. In refractory asthma, medications cannot reverse the tightening and swelling of the airways.

There is a need to develop methods of treating or preventing COPD; or at least the provision of alternative methods to compliment the previously known methods to improve the symptoms of COPD. Therefore, the present invention seeks to provide an improved or alternative method for the treatment or prevention of COPD.

The previous discussion of the background art is intended to facilitate an understanding of the present invention only. The discussion is not an acknowledgement or admission that any of the material referred to is or was part of the common general knowledge as at the priority date of the application.

### SUMMARY OF INVENTION

The present invention provides a pharmaceutical formulation comprising:
a) at least one angiotensin type 1 receptor (AT₁R) blocker; and
b) at least one CXC chemokine receptor 2 (CXCR2) pathway inhibitor.

Preferably the formulation is for use in the treatment, amelioration or prevention of a condition or disease that is COPD. Preferably the COPD is selected from: emphysema, chronic bronchitis, bronchiectasis, and refractory (non-reversible) asthma.

The CXCR2 inhibitor and the AT₁R blocker may be administered in the same dosage form or in separate dosage forms.

The CXCR2 inhibitor and the AT₁R blocker may be administered concurrently or sequentially.

CXCR2 inhibitors include pharmaceutically acceptable salts of CXCR2 inhibitors and antibody inhibitors of the CXCR2. AT₁R blockers include pharmaceutically acceptable salts of AT₁R blockers and antibody blockers of the AT₁R. The CXCR2 inhibitor and the AT₁R blocker may be the same active agent, for example a bi-specific antibody. Preferably the CXCR2 pathway inhibitor is a direct CXCR2 antagonist, negative allosteric CXCR2 modulator, CXCR2 inverse agonist or allosteric inverse agonist.

The invention further provides a method for the treatment, amelioration or prevention of a condition or disease, said method comprising the step of:
i) administering to a subject a therapeutically effective amount of a combination of (a) an angiotensin type 1 receptor (AT₁R) blocker and (b) a CXC chemokine receptor 2 (CXCR2) pathway inhibitor.

The invention also provides for the use of (a) at least one angiotensin type 1 receptor (AT₁R) blocker, and (b) at least one CXC chemokine receptor 2 (CXCR2) pathway inhibitor; for the manufacture of a dosage form for the treatment or prevention of a condition or disease.

The present invention provides a kit for the treatment, amelioration or prevention of a condition or disease, said kit comprising:
a) at least one angiotensin type 1 receptor (AT₁R) blocker;
b) at least one CXC chemokine receptor 2 (CXCR2) pathway inhibitor; and
c) instructions for use.

The present invention provides at least one AT₁R blocker, and at least one CXCR2 inhibitor, for use in a formulation for the treatment, amelioration or prevention of a disease.

The present invention provides at least one AT₁R blocker for use in a formulation for the treatment, amelioration or prevention of a disease wherein the at least one AT₁R blocker is administered to the subject concurrently or sequentially with at least one CXCR2 inhibitor.

The present invention provides at least one CXCR2 inhibitor for use in a formulation for the treatment, amelioration or prevention of a disease wherein the at least one CXCR2 inhibitor is administered to the subject concurrently or sequentially with at least one AT₁R blocker.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. Noting that all bioluminescence resonance energy transfer (BRET) signals were measured at 37° C. from transiently-transfected Human Embryonic Kidney (HEK) 293FT cells and in these experiments pcDNA3 was co-transfected where required to keep cDNA levels the same, the description will be made with reference to the accompanying drawings in which:

FIG. 1A shows BRET signals from cells expressing CXCR2/Rluc8 (CXCR2 labelled with Rluc8) and Venus/mGsi (mGsi as a sensor for Gi activity labelled with Venus) without hemagglutinin epitope-tagged AT₁R (HA-AT₁R) expressed following treatment with either 10⁻⁷M (100 nM) CXCL8 or 10⁻⁶M (1 μM) AngII only or both CXCL8 and AngII combined.

FIG. 1B shows BRET signals from cells expressing CXCR2/Rluc8, Venus/mGsi and HA-AT1R following treatment with either 10⁻⁷M (100 nM) CXCL8 or 10⁻⁶M (1 μM) AngII only or both CXCL8 and AngII combined.

FIG. 2A shows, in HEK293 cells transiently-transfected with AT1R cDNA (750 ng) and CXCR2 cDNA (250 ng), concentration-response curves for D-myo-Inositol 1-Phosphate (IP1) accumulation following treatment with either a range of CXCL8 concentrations as indicated or a range of AngII concentrations as indicated or a range of AngII concentrations as indicated in combination with 10 nM CXCL8.

FIG. 2B shows, in HEK293 cells stably-expressing CXCR2 and transiently transfected with AT1R cDNA (500 ng), concentration-response curves for D-myo-Inositol 1-Phosphate (IP1) accumulation following treatment with either a range of CXCL8 concentrations as indicated or a range of AngII concentrations as indicated or a range of AngII concentrations as indicated in combination with 1 nM CXCL8.

FIG. 3 shows a simplified schematic representation of subcellular marker localization and receptor trafficking (published in Tiulpakov et al (2016) Mol. Endocrinol. 30, 889-904). Ligand-induced trafficking was monitored using Rluc8-tagged proteins of interest by measuring proximity via BRET with the plasma membrane marker Venus/Kras (K-ras), or the subcellular compartment markers Rabs: Venus/Rab5 (5) for early endosomes; Venus/Rab4 (4) for early endosome recycling; Venus/Rab11a (11) for recycling endosomes; Venus/Rab7 (7) for late endosomes/lysosomes; Venus/Rab9 (9) for late endosome trafficking to the trans-Golgi network; Venus/Rab1 (1) for endoplasmic reticulum trafficking to the cis-Golgi; Venus/Rab6 (6) for Golgi apparatus and trans-Golgi network; or Venus/Rab8 (8) for trans-Golgi network to plasma membrane.

FIG. 4A shows BRET signals from cells expressing CXCR2/Rluc8 and Venus/Kras without HA-AT1R following treatment with either 100 nM CXCL8 or 1 μM AngII only or both CXCL8 and AngII combined.

FIG. 4B shows BRET signals from cells expressing CXCR2/Rluc8, Venus/Kras and HA-AT1R following treatment with either 100 nM CXCL8 or 1 μM AngII only or both CXCL8 and AngII combined.

FIG. 4C shows BRET signals from cells expressing CXCR2/Rluc8 and Venus/Rab1 without HA-AT1R following treatment with either 100 nM CXCL8 or 1 μM AngII only or both CXCL8 and AngII combined.

FIG. 4D shows BRET signals from cells expressing CXCR2/Rluc8, Venus/Rab1 and HA-AT1R following treatment with either 100 nM CXCL8 or 1 μM AngII only or both CXCL8 and AngII combined.

FIG. 4E shows BRET signals from cells expressing CXCR2/Rluc8 and Venus/Rab4 without HA-AT1R following treatment with either 100 nM CXCL8 or 1 μM AngII only or both CXCL8 and AngII combined.

FIG. 4F shows BRET signals from cells expressing CXCR2/Rluc8, Venus/Rab4 and HA-AT1R following treatment with either 100 nM CXCL8 or 1 μM AngII only or both CXCL8 and AngII combined.

FIG. 4G shows BRET signals from cells expressing CXCR2/Rluc8 and Venus/Rab5 without HA-AT1R following treatment with either 100 nM CXCL8 or 1 μM AngII only or both CXCL8 and AngII combined.

FIG. 4H shows BRET signals from cells expressing CXCR2/Rluc8, Venus/Rab5 and HA-AT1R following treatment with either 100 nM CXCL8 or 1 μM AngII only or both CXCL8 and AngII combined.

FIG. 4I shows BRET signals from cells expressing CXCR2/Rluc8 and Venus/Rab6 without HA-AT1R following treatment with either 100 nM CXCL8 or 1 μM AngII only or both CXCL8 and AngII combined.

FIG. 4J shows BRET signals from cells expressing CXCR2/Rluc8, Venus/Rab6 and HA-AT1R following treatment with either 100 nM CXCL8 or 1 μM AngII only or both CXCL8 and AngII combined.

FIG. 4K shows BRET signals from cells expressing CXCR2/Rluc8 and Venus/Rab7 without HA-AT1R following treatment with either 100 nM CXCL8 or 1 μM AngII only or both CXCL8 and AngII combined.

FIG. 4L shows BRET signals from cells expressing CXCR2/Rluc8, Venus/Rab7 and HA-AT1R following treatment with either 100 nM CXCL8 or 1 μM AngII only or both CXCL8 and AngII combined.

FIG. 4M shows BRET signals from cells expressing CXCR2/Rluc8 and Venus/Rab8 without HA-AT1R following treatment with either 100 nM CXCL8 or 1 μM AngII only or both CXCL8 and AngII combined.

FIG. 4N shows BRET signals from cells expressing CXCR2/Rluc8, Venus/Rab8 and HA-AT1R following treatment with either 100 nM CXCL8 or 1 μM AngII only or both CXCL8 and AngII combined.

FIG. 4O shows BRET signals from cells expressing CXCR2/Rluc8 and Venus/Rab9 without HA-AT1R following treatment with either 100 nM CXCL8 or 1 μM AngII only or both CXCL8 and AngII combined.

FIG. 4P shows BRET signals from cells expressing CXCR2/Rluc8, Venus/Rab9 and HA-AT1R following treatment with either 100 nM CXCL8 or 1 μM AngII only or both CXCL8 and AngII combined.

FIG. 4Q shows BRET signals from cells expressing CXCR2/Rluc8 and Venus/Rab11a without HA-AT1R following treatment with either 100 nM CXCL8 or 1 μM AngII only or both CXCL8 and AngII combined.

FIG. 4R shows BRET signals from cells expressing CXCR2/Rluc8, Venus/Rab11a and HA-AT1R following treatment with either 100 nM CXCL8 or 1 μM AngII only or both CXCL8 and AngII combined.

FIG. 5A shows BRET signals from cells expressing AT1R/Rluc8 and Venus/Kras without unlabelled CXCR2 following treatment with either 100 nM CXCL8 or 1 μM AngII only or both CXCL8 and AngII combined.

FIG. 5B shows BRET signals from cells expressing AT1R/Rluc8, Venus/Kras and unlabelled CXCR2 following treatment with either 100 nM CXCL8 or 1 μM AngII only or both CXCL8 and AngII combined.

FIG. 5C shows BRET signals from cells expressing AT1R/Rluc8 and Venus/Rab1 without unlabelled CXCR2 following treatment with either 100 nM CXCL8 or 1 μM AngII only or both CXCL8 and AngII combined.

FIG. 5D shows BRET signals from cells expressing AT1R/Rluc8, Venus/Rab1 and unlabelled CXCR2 following treatment with either 100 nM CXCL8 or 1 μM AngII only or both CXCL8 and AngII combined.

FIG. 5E shows BRET signals from cells expressing AT1R/Rluc8 and Venus/Rab4 without unlabelled CXCR2 following treatment with either 100 nM CXCL8 or 1 μM AngII only or both CXCL8 and AngII combined.

FIG. 5F shows BRET signals from cells expressing AT1R/Rluc8, Venus/Rab4 and unlabelled CXCR2 following treatment with either 100 nM CXCL8 or 1 μM AngII only or both CXCL8 and AngII combined.

FIG. 5G shows BRET signals from cells expressing AT1R/Rluc8 and Venus/Rab5 without unlabelled CXCR2 following treatment with either 100 nM CXCL8 or 1 μM AngII only or both CXCL8 and AngII combined.

FIG. 5H shows BRET signals from cells expressing AT1R/Rluc8, Venus/Rab5 and unlabelled CXCR2 following treatment with either 100 nM CXCL8 or 1 μM AngII only or both CXCL8 and AngII combined.

FIG. 5I shows BRET signals from cells expressing AT1R/Rluc8 and Venus/Rab6 without unlabelled CXCR2 following treatment with either 100 nM CXCL8 or 1 μM AngII only or both CXCL8 and AngII combined.

FIG. 5J shows BRET signals from cells expressing AT1R/Rluc8, Venus/Rab6 and unlabelled CXCR2 following treatment with either 100 nM CXCL8 or 1 μM AngII only or both CXCL8 and AngII combined.

FIG. 5K shows BRET signals from cells expressing AT1R/Rluc8 and Venus/Rab7 without unlabelled CXCR2 following treatment with either 100 nM CXCL8 or 1 μM AngII only or both CXCL8 and AngII combined.

FIG. 5L shows BRET signals from cells expressing AT1R/Rluc8, Venus/Rab7 and unlabelled CXCR2 following treatment with either 100 nM CXCL8 or 1 μM AngII only or both CXCL8 and AngII combined.

FIG. 5M shows BRET signals from cells expressing AT1R/Rluc8 and Venus/Rab8 without unlabelled CXCR2 following treatment with either 100 nM CXCL8 or 1 μM AngII only or both CXCL8 and AngII combined.

FIG. 5N shows BRET signals from cells expressing AT1R/Rluc8, Venus/Rab8 and unlabelled CXCR2 following treatment with either 100 nM CXCL8 or 1 μM AngII only or both CXCL8 and AngII combined.

FIG. 5O shows BRET signals from cells expressing AT1R/Rluc8 and Venus/Rab9 without unlabelled CXCR2 following treatment with either 100 nM CXCL8 or 1 μM AngII only or both CXCL8 and AngII combined.

FIG. 5P shows BRET signals from cells expressing AT1R/Rluc8, Venus/Rab9 and unlabelled CXCR2 following treatment with either 100 nM CXCL8 or 1 μM AngII only or both CXCL8 and AngII combined.

FIG. 5Q shows BRET signals from cells expressing AT1R/Rluc8 and Venus/Rab11a without unlabelled CXCR2 following treatment with either 100 nM CXCL8 or 1 μM AngII only or both CXCL8 and AngII combined.

FIG. 5R shows BRET signals from cells expressing AT1R/Rluc8, Venus/Rab11a and unlabelled CXCR2 following treatment with either 100 nM CXCL8 or 1 μM AngII only or both CXCL8 and AngII combined.

FIG. 6A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with vehicle.

FIG. 6B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with CXCR2 allosteric inverse agonist SB265610.

FIG. 6C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with CXCR2 antagonist SB225002.

FIG. 6D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with CXCR2 inverse agonist navarixin (SCH527123).

FIG. 6E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with CXCR2 antagonist AZD5069.

FIG. 6F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with CXCR2 antagonist danirixin.

FIG. 7A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist irbesartan.

FIG. 7B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist irbesartan and CXCR2 allosteric inverse agonist SB265610 combined.

FIG. 7C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist irbesartan and CXCR2 antagonist SB225002 combined.

FIG. 7D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist irbesartan and CXCR2 inverse agonist navarixin (SCH527123) combined.

FIG. 7E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist irbesartan and CXCR2 antagonist AZD5069 combined.

FIG. 7F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist irbesartan and CXCR2 antagonist danirixin combined.

FIG. 8A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist olmesartan.

FIG. 8B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist olmesartan and CXCR2 allosteric inverse agonist SB265610 combined.

FIG. 8C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist olmesartan and CXCR2 antagonist SB225002 combined.

FIG. 8D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist olmesartan and CXCR2 inverse agonist navarixin (SCH527123) combined.

FIG. 8E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist olmesartan and CXCR2 antagonist AZD5069 combined.

FIG. 8F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist olmesartan and CXCR2 antagonist danirixin combined.

FIG. 9A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist candesartan.

FIG. 9B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist candesartan and CXCR2 allosteric inverse agonist SB265610 combined.

FIG. 9C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist candesartan and CXCR2 antagonist SB225002 combined.

FIG. 9D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist candesartan and CXCR2 inverse agonist navarixin (SCH527123) combined.

FIG. 9E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist candesartan and CXCR2 antagonist AZD5069 combined.

FIG. 9F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist candesartan and CXCR2 antagonist danirixin combined.

FIG. 10A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist valsartan.

FIG. 10B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist valsartan and CXCR2 allosteric inverse agonist SB265610 combined.

FIG. 10C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist valsartan and CXCR2 antagonist SB225002 combined.

FIG. 10D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist valsartan and CXCR2 inverse agonist navarixin (SCH527123) combined.

FIG. 10E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist valsartan and CXCR2 antagonist AZD5069 combined.

FIG. 10F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist valsartan and CXCR2 antagonist danirixin combined.

FIG. 11A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist eprosartan.

FIG. 11B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist eprosartan and CXCR2 allosteric inverse agonist SB265610 combined.

FIG. 11C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist eprosartan and CXCR2 antagonist SB225002 combined.

FIG. 11D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist eprosartan and CXCR2 inverse agonist navarixin (SCH527123) combined.

FIG. 11E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist eprosartan and CXCR2 antagonist AZD5069 combined.

FIG. 11F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist eprosartan and CXCR2 antagonist danirixin combined.

FIG. 12A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist azilsartan.

FIG. 12B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist azilsartan and CXCR2 allosteric inverse agonist SB265610 combined.

FIG. 12C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist azilsartan and CXCR2 antagonist SB225002 combined.

FIG. 12D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist azilsartan and CXCR2 inverse agonist navarixin (SCH527123) combined.

FIG. 12E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist azilsartan and CXCR2 antagonist AZD5069 combined.

FIG. 12F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist azilsartan and CXCR2 antagonist danirixin combined.

FIG. 13A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist losartan.

FIG. 13B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist losartan and CXCR2 allosteric inverse agonist SB265610 combined.

FIG. 13C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist losartan and CXCR2 antagonist SB225002 combined.

FIG. 13D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist losartan and CXCR2 inverse agonist navarixin (SCH527123) combined.

FIG. 13E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist losartan and CXCR2 antagonist AZD5069 combined.

FIG. 13F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist losartan and CXCR2 antagonist danirixin combined.

FIG. 14A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist EXP3174 (active metabolite of losartan).

FIG. 14B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist EXP3174 (active metabolite of losartan) and CXCR2 allosteric inverse agonist SB265610 combined.

FIG. 14C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist EXP3174 (active metabolite of losartan) and CXCR2 antagonist SB225002 combined.

FIG. 14D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist EXP3174 (active metabolite of losartan) and CXCR2 inverse agonist navarixin (SCH527123) combined.

FIG. 14E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist EXP3174 (active metabolite of losartan) and CXCR2 antagonist AZD5069 combined.

FIG. 14F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist EXP3174 (active metabolite of losartan) and CXCR2 antagonist danirixin combined.

FIG. 15A shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with AT1R antagonist telmisartan.

FIG. 15B shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist telmisartan and CXCR2 allosteric inverse agonist SB265610 combined.

FIG. 15C shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist telmisartan and CXCR2 antagonist SB225002 combined.

FIG. 15D shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist telmisartan and CXCR2 inverse agonist navarixin (SCH527123) combined.

FIG. 15E shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist telmisartan and CXCR2 antagonist AZD5069 combined.

FIG. 15F shows BRET signals from cells expressing CXCR2/Rluc8, Venus/mGsi and HA-AT1R following treatment with vehicle or 10 nM CXCL8 only or 100 nM AngII only or both CXCL8 and AngII combined, 50 minutes after pre-treatment with both AT1R antagonist telmisartan and CXCR2 antagonist danirixin combined.

FIG. 16A shows BRET signals from cells expressing CXCR2/Rluc8, Venus/Kras and HA-AT1R following treatment with vehicle or 10 nM CXCL8 only or 100 nM AngII only or both CXCL8 and AngII combined, 50 minutes after pre-treatment with vehicle.

FIG. 16B shows BRET signals from cells expressing CXCR2/Rluc8, Venus/Kras and HA-AT1R following treatment with 100 nM AngII only, 50 minutes after pre-treatment with AT1R antagonist irbesartan (Irb), valsartan (Val) or azilsartan (Azil).

FIG. 16C shows BRET signals from cells expressing CXCR2/Rluc8, Venus/Kras and HA-AT1R following treatment with both 10 nM CXCL8 and 100 nM AngII combined, 50 minutes after pre-treatment with AT1R antagonist irbesartan (Irb), valsartan (Val) or azilsartan (Azil).

FIG. 16D shows BRET signals from cells expressing CXCR2/Rluc8, Venus/Kras and HA-AT1R following treatment with 100 nM AngII only, 50 minutes after pre-treatment with CXCR2 inverse agonist navarixin (SCH527123; SCH), antagonist SB225002 (SB22) or allosteric inverse agonist SB265610 (SB26).

FIG. 16E shows BRET signals from cells expressing CXCR2/Rluc8, Venus/Kras and HA-AT1R following treatment with both 10 nM CXCL8 and 100 nM AngII combined, 50 minutes after pre-treatment with CXCR2 inverse agonist navarixin (SCH527123; SCH), antagonist SB225002 (SB22) or allosteric inverse agonist SB265610 (SB26).

FIG. 16F shows BRET signals from cells expressing CXCR2/Rluc8, Venus/Kras and HA-AT1R following treatment with both 10 nM CXCL8 and 100 nM AngII combined, 50 minutes after pre-treatment with both CXCR2 inverse agonist navarixin (SCH527123; SCH) and either AT1R antagonist irbesartan (Irb), valsartan (Val) or azilsartan (Azil).

FIG. 16G shows BRET signals from cells expressing CXCR2/Rluc8, Venus/Kras and HA-AT1R following treatment with both 10 nM CXCL8 and 100 nM AngII combined, 50 minutes after pre-treatment with both CXCR2 antagonist SB225002 (SB22) and either AT1R antagonist irbesartan (Irb), valsartan (Val) or azilsartan (Azil).

FIG. 16H shows BRET signals from cells expressing CXCR2/Rluc8, Venus/Kras and HA-AT1R following treatment with both 10 nM CXCL8 and 100 nM AngII combined, 50 minutes after pre-treatment with both CXCR2 allosteric inverse agonist SB265610 (SB26) and either AT1R antagonist irbesartan (Irb), valsartan (Val) or azilsartan (Azil).

FIG. 17A shows BRET signals from cells expressing CXCR2/Rluc8, Venus/Rab5 and HA-AT1R following treatment with vehicle or 10 nM CXCL8 only or 100 nM AngII only or both CXCL8 and AngII combined, 50 minutes after pre-treatment with vehicle.

FIG. 17B shows BRET signals from cells expressing CXCR2/Rluc8, Venus/Rab5 and HA-AT1R following treatment with 100 nM AngII only, 50 minutes after pre-treatment with AT1R antagonist irbesartan (Irb), valsartan (Val) or azilsartan (Azil).

FIG. 17C shows BRET signals from cells expressing CXCR2/Rluc8, Venus/Rab5 and HA-AT1R following treatment with both 10 nM CXCL8 and 100 nM AngII combined, 50 minutes after pre-treatment with AT1R antagonist irbesartan (Irb), valsartan (Val) or azilsartan (Azil).

FIG. 17D shows BRET signals from cells expressing CXCR2/Rluc8, Venus/Rab5 and HA-AT1R following treatment with 100 nM AngII only, 50 minutes after pre-treatment with CXCR2 inverse agonist navarixin (SCH527123; SCH), antagonist SB225002 (SB22) or allosteric inverse agonist SB265610 (SB26).

FIG. 17E shows BRET signals from cells expressing CXCR2/Rluc8, Venus/Rab5 and HA-AT1R following treatment with both 10 nM CXCL8 and 100 nM AngII combined, 50 minutes after pre-treatment with CXCR2 inverse agonist navarixin (SCH527123; SCH), antagonist SB225002 (SB22) or allosteric inverse agonist SB265610 (SB26).

FIG. 17F shows BRET signals from cells expressing CXCR2/Rluc8, Venus/Rab5 and HA-AT1R following treatment with both 10 nM CXCL8 and 100 nM AngII combined, 50 minutes after pre-treatment with both CXCR2 inverse agonist navarixin (SCH527123; SCH) and either AT1R antagonist irbesartan (Irb), valsartan (Val) or azilsartan (Azil).

FIG. 17G shows BRET signals from cells expressing CXCR2/Rluc8, Venus/Rab5 and HA-AT1R following treatment with both 10 nM CXCL8 and 100 nM AngII combined, 50 minutes after pre-treatment with both CXCR2 antagonist SB225002 (SB22) and either AT1R antagonist irbesartan (Irb), valsartan (Val) or azilsartan (Azil).

FIG. 17H shows BRET signals from cells expressing CXCR2/Rluc8, Venus/Rab5 and HA-AT1R following treatment with both 10 nM CXCL8 and 100 nM AngII combined, 50 minutes after pre-treatment with both CXCR2 allosteric inverse agonist SB265610 (SB26) and either AT1R antagonist irbesartan (Irb), valsartan (Val) or azilsartan (Azil).

FIG. 18A shows BRET signals from cells expressing CXCR2/Rluc8, Venus/Rab8 and HA-AT1R following treatment with vehicle or 10 nM CXCL8 only or 100 nM AngII only or both CXCL8 and AngII combined, 50 minutes after pre-treatment with vehicle.

FIG. 18B shows BRET signals from cells expressing CXCR2/Rluc8, Venus/Rab8 and HA-AT1R following treatment with 100 nM AngII only, 50 minutes after pre-treatment with AT1R antagonist irbesartan (Irb), valsartan (Val) or azilsartan (Azil).

FIG. 18C shows BRET signals from cells expressing CXCR2/Rluc8, Venus/Rab8 and HA-AT1R following treatment with both 10 nM CXCL8 and 100 nM AngII combined, 50 minutes after pre-treatment with AT1R antagonist irbesartan (Irb), valsartan (Val) or azilsartan (Azil).

FIG. 18D shows BRET signals from cells expressing CXCR2/Rluc8, Venus/Rab8 and HA-AT1R following treatment with 100 nM AngII only, 50 minutes after pre-treatment with CXCR2 inverse agonist navarixin (SCH527123; SCH), antagonist SB225002 (SB22) or allosteric inverse agonist SB265610 (SB26).

FIG. 18E shows BRET signals from cells expressing CXCR2/Rluc8, Venus/Rab8 and HA-AT1R following treatment with both 10 nM CXCL8 and 100 nM AngII combined, 50 minutes after pre-treatment with CXCR2 inverse agonist navarixin (SCH527123; SCH), antagonist SB225002 (SB22) or allosteric inverse agonist SB265610 (SB26).

FIG. 18F shows BRET signals from cells expressing CXCR2/Rluc8, Venus/Rab8 and HA-AT1R following treatment with both 10 nM CXCL8 and 100 nM AngII combined, 50 minutes after pre-treatment with both CXCR2 inverse agonist navarixin (SCH527123; SCH) and either AT1R antagonist irbesartan (Irb), valsartan (Val) or azilsartan (Azil).

FIG. 18G shows BRET signals from cells expressing CXCR2/Rluc8, Venus/Rab8 and HA-AT1R following treatment with both 10 nM CXCL8 and 100 nM AngII combined, 50 minutes after pre-treatment with both CXCR2 antagonist SB225002 (SB22) and either AT1R antagonist irbesartan (Irb), valsartan (Val) or azilsartan (Azil).

FIG. 18H shows BRET signals from cells expressing CXCR2/Rluc8, Venus/Rab8 and HA-AT1R following treatment with both 10 nM CXCL8 and 100 nM AngII combined, 50 minutes after pre-treatment with both CXCR2 allosteric inverse agonist SB265610 (SB26) and either AT1R antagonist irbesartan (Irb), valsartan (Val) or azilsartan (Azil).

FIG. 19A shows BRET signals from cells expressing AT1R/Rluc8, Venus/Kras and unlabelled CXCR2 following treatment with vehicle or 100 nM AngII only, 50 minutes after pre-treatment with vehicle or either AT1R antagonist irbesartan (Irb), valsartan (Val) or azilsartan (Azil).

FIG. 19B shows BRET signals from cells expressing AT1R/Rluc8, Venus/Kras and unlabelled CXCR2 following treatment with both 10 nM CXCL8 and 100 nM AngII combined, 50 minutes after pre-treatment with vehicle or either AT1R antagonist irbesartan (Irb), valsartan (Val) or azilsartan (Azil).

FIG. 19C shows BRET signals from cells expressing AT1R/Rluc8, Venus/Kras and unlabelled CXCR2 following treatment with both 10 nM CXCL8 and 100 nM AngII combined, 50 minutes after pre-treatment with CXCR2 inverse agonist navarixin (SCH527123; SCH), antagonist SB225002 (SB22), allosteric inverse agonist SB265610 (SB26), antagonist AZD5069 (AZD) or antagonist Danirixin (Dnrx).

FIG. 19D shows BRET signals from cells expressing AT1R/Rluc8, Venus/Kras and unlabelled CXCR2 following treatment with both 10 nM CXCL8 and 100 nM AngII combined, 50 minutes after pre-treatment with both CXCR2 inverse agonist navarixin (SCH527123; SCH) and either AT1R antagonist irbesartan (Irb), valsartan (Val) or azilsartan (Azil).

FIG. 19E shows BRET signals from cells expressing AT1R/Rluc8, Venus/Kras and unlabelled CXCR2 following treatment with both 10 nM CXCL8 and 100 nM AngII combined, 50 minutes after pre-treatment with both CXCR2 antagonist SB225002 (SB22) and either AT1R antagonist irbesartan (Irb), valsartan (Val) or azilsartan (Azil).

FIG. 19F shows BRET signals from cells expressing AT1R/Rluc8, Venus/Kras and unlabelled CXCR2 following treatment with both 10 nM CXCL8 and 100 nM AngII combined, 50 minutes after pre-treatment with both CXCR2 allosteric inverse agonist SB265610 (SB26) and either AT1R antagonist irbesartan (Irb), valsartan (Val) or azilsartan (Azil).

FIG. 20A shows BRET signals from cells expressing AT1R/Rluc8, Venus/Rab5 and unlabelled CXCR2 following treatment with vehicle or 100 nM AngII only, 50 minutes after pre-treatment with vehicle or either AT1R antagonist irbesartan (Irb), valsartan (Val) or azilsartan (Azil).

FIG. 20B shows BRET signals from cells expressing AT1R/Rluc8, Venus/Rab5 and unlabelled CXCR2 following treatment with both 10 nM CXCL8 and 100 nM AngII combined, 50 minutes after pre-treatment with vehicle or either AT1R antagonist irbesartan (Irb), valsartan (Val) or azilsartan (Azil).

FIG. 20C shows BRET signals from cells expressing AT1R/Rluc8, Venus/Rab5 and unlabelled CXCR2 following treatment with both 10 nM CXCL8 and 100 nM AngII combined, 50 minutes after pre-treatment with CXCR2 inverse agonist navarixin (SCH527123; SCH), antagonist SB225002 (SB22), allosteric inverse agonist SB265610 (SB26), antagonist AZD5069 (AZD) or antagonist Danirixin (Dnrx).

FIG. 20D shows BRET signals from cells expressing AT1R/Rluc8, Venus/Rab5 and unlabelled CXCR2 following treatment with both 10 nM CXCL8 and 100 nM AngII combined, 50 minutes after pre-treatment with both CXCR2 inverse agonist navarixin (SCH527123; SCH) and either AT1R antagonist irbesartan (Irb), valsartan (Val) or azilsartan (Azil).

FIG. 20E shows BRET signals from cells expressing AT1R/Rluc8, Venus/Rab5 and unlabelled CXCR2 following treatment with both 10 nM CXCL8 and 100 nM AngII combined, 50 minutes after pre-treatment with both CXCR2 antagonist SB225002 (SB22) and either AT1R antagonist irbesartan (Irb), valsartan (Val) or azilsartan (Azil).

FIG. 20F shows BRET signals from cells expressing AT1R/Rluc8, Venus/Rab5 and unlabelled CXCR2 following treatment with both 10 nM CXCL8 and 100 nM AngII combined, 50 minutes after pre-treatment with both CXCR2 allosteric inverse agonist SB265610 (SB26) and either AT1R antagonist irbesartan (Irb), valsartan (Val) or azilsartan (Azil).

### DESCRIPTION OF INVENTION

### Detailed Description of the Invention

The present invention has unexpectedly identified a heteromer association between the angiotensin type 1 receptor (AT₁R; AT1R) and CXC chemokine receptor 2 (CXCR2). Both of these receptors have been independently implicated in the pathophysiology of COPD (Traves et al (2004) Specific CXC but not CC chemokines cause elevated monocyte migration in COPD: a role for CXCR2. J. Leukoc. Biol. 76, 441-450). It has previously been found that CXCR2 reduces neutrophil chemotaxis and reduces mucus production and airway inflammation in chronic obstructive pulmonary disease (COPD). However, the efficacy of CXCR2 inhibitors in treatment of COPD has been disappointing during clinical studies.

Without being held to any theory, it is believed that the inability of CXCR2 inhibitors to treat COPD is due to the heteromeric nature of CXCR2 and AT₁R.

### Composition

The present invention provides a pharmaceutical formulation comprising:
a) at least one angiotensin type 1 receptor (AT₁R) blocker; and
b) at least one CXC chemokine receptor 2 (CXCR2) pathway inhibitor.

The AT₁R blocker inhibits or partially inhibits the AT₁R. Preferably, the AT₁R blocker directly interacts with the AT₁R, and does not include therapeutics such as angiotensin converting enzyme inhibitors (ACEi) therapeutics that act upstream of the AT₁R to prevent generation of the functional ligand but that do not interact with the AT₁R itself.

The term “AT₁R blocker” includes pharmaceutically acceptable salts of AT₁R blockers. Alternatively, the AT₁R blocker may be an antibody blocker of the AT₁R.

The CXCR2 pathway inhibitor inhibits or partially inhibits the CXCR2 and/or the CXCR2 signalling pathway. For example, the CXCR2 pathway inhibitor may include any compound or agent which inhibits or partially inhibits any one of the pathways associated with signalling of the CXCR2, including compounds or agents which inhibit components of the CXCR2 pathway other than the chemokine receptor itself. The CXCR2 pathway inhibitor may be, but are not limited to, an antagonist of CXCR2 or components of the CXCR2 pathway other than CXCR2, an inverse agonist of CXCR2 or components of the CXCR2 pathway other than CXCR2 or a negative allosteric modulator of CXCR2 or components of the CXCR2 pathway other than CXCR2.

An inverse agonist of CXCR2 can also be a negative allosteric modulator of CXCR2. In such a case it can be referred to as a CXCR2 allosteric inverse agonist.

A CXCR2 inhibitor can act as an antagonist or inverse agonist for different signalling pathways. For example, a CXCR2 inhibitor can be a CXCR2 antagonist for one signalling pathway and a CXCR2 inverse agonist for another signalling pathway.

A CXCR2 inverse agonist may be classified as a CXCR2 antagonist where constitutive activity is not apparent for the assessed signalling pathway in order to enable definition as an inverse agonist.

A CXCR2 negative allosteric modulator may be classified as a CXCR2 antagonist if it inhibits CXCR2 but the binding mode has not been clearly defined (orthosteric versus allosteric) or appreciated.

Preferably the CXCR2 pathway inhibitor directly inhibits the receptor itself, rather than an upstream or downstream component of the CXCR2 pathway.

The term “CXCR2 inhibitor” includes pharmaceutically acceptable salts of CXCR2 inhibitors. Alternatively, the CXCR2 inhibitor may be an antibody inhibitor of the CXCR2.

The CXCR2 inhibitor and the AT₁R blocker may be the same active agent, for example a bi-specific antibody.

The pharmaceutical formulation may optionally include excipients, solvents, carriers and other pharmaceutically acceptable ingredients.

The term “component” as used herein in the context of a pharmaceutical formulation of the invention, means either the AT₁R blocker or the CXCR2 pathway inhibitor.

The term “inhibits” as used herein, means a reduction below detectable limits when compared to a reference. The phrase includes blocking, retarding, or impeding an action to prevent an undesirable result.

The term “partially inhibits” as used herein, means any reduction within detectable limits when compared to a reference. The phrase includes blocking, retarding, or impeding an action to prevent an undesirable result.

### CXC Chemokine Receptor 2

The CXC chemokine receptor 2 (CXCR2) is a G-protein coupled receptor.

The phrase “CXCR2 pathway inhibitor” is intended to include any compound or agent which inhibits or partially inhibits any one of the pathways associated with the CXCR2, including compounds or agents which inhibit components of the CXCR2 pathway other than the chemokine receptor itself. For example, the inhibitor may inhibit or partially inhibit proteins that associate with CXCR2, or may inhibit compounds or pathway steps before and/or after CXCR2 itself. Preferably, the CXCR2 pathway inhibitor is a CXCR2 antagonist, CXCR2 inverse agonist or CXCR2 negative allosteric modulator.

The term “a component of the CXCR2 pathway other than CXCR2” as used herein, is to be understood as including a component of any one of the pathways listed above which is triggered by CXCR2, wherein the component is itself not CXCR2. Preferably, the component is a protein such as, but not limited to, a transduction or signalling protein. The component of the chemokine receptor pathway may interact directly with CXCR2. Alternatively, the component of the chemokine receptor pathway may interact indirectly with CXCR2 by way of protein-protein interaction or complex formation. Alternatively, the component of the chemokine receptor pathway may interact indirectly with CXCR2 by way of a signalling cascade such as is known in the art.

The CXCR2 pathway inhibitor may be selected from the group comprising: a direct CXCR2 antagonist, an inverse CXCR2 agonist, a negative allosteric CXCR2 modulator, an allosteric inverse CXCR2 agonist, an indirect CXCR2 antagonist, an indirect inverse CXCR2 agonist, and an indirect negative allosteric CXCR2 modulator. Preferably, the CXCR2 pathway inhibitor is a CXCR2 antagonist, CXCR2 inverse agonist, CXCR2 negative allosteric modulator or CXCR2 allosteric inverse agonist. Preferably the CXCR2 pathway inhibitor is a direct CXCR2 antagonist, negative allosteric CXCR2 modulator, CXCR2 inverse agonist or CXCR2 allosteric inverse agonist.

In one preferred embodiment the chemokine receptor pathway inhibitor is selected from the group consisting of:
i) antagonists of CXCR2 or components of the CXCR2 pathway other than CXCR2;
ii) inverse agonists of CXCR2 or components of the CXCR2 pathway other than CXCR2;
iii) negative allosteric modulators of CXCR2 or components of the CXCR2 pathway other than CXCR2.

The term “CXCR2 inhibitor” includes pharmaceutically acceptable salts of CXCR2 inhibitors. Alternatively, the CXCR2 inhibitor may be an antibody inhibitor of the CXCR2.

Known antagonists of CXCR2 include: repertaxin (reparixin), danirixin (GSK1325756), AZD5069, SB225002 and elubrixin.

Known inverse agonists of CXCR2 include: SB265610 and navarixin (MK-7123; SCH527123).

Known negative allosteric modulators of CXCR2 include: SB265610.

In one preferred embodiment the CXCR2 pathway inhibitor is an antagonist of CXCR2. For example, the CXCR2 pathway inhibitor may be selected from the group comprising: repertaxin, danirixin (GSK1325756), AZD5069, SB225002 and elubrixin.

In one preferred embodiment the CXCR2 pathway inhibitor is an inverse agonist of CXCR2. For example, the CXCR2 pathway inhibitor may be selected from the group comprising: SB265610 (Bradley et al (2009) Br. J. Pharmacol. 158, 328-338) and navarixin (MK-7123; SCH527123; Kredel et al (2009) J Biol Screen 14, 1076-1091).

In one preferred embodiment the CXCR2 pathway inhibitor is a negative allosteric modulator of CXCR2. For example, the CXCR2 pathway inhibitor may be selected from the group comprising: SB265610 (Bradley et al (2009) Br. J. Pharmacol. 158, 328-338).

### Angiotensin Type 1 Receptor

The angiotensin type 1 receptor (AT₁R, AT₁R, Angiotensin II receptor type 1) is a G protein-coupled receptor.

The phrase “angiotensin type 1 receptor inhibitor” (also referred to as an angiotensin receptor blocker or ARB) is understood to mean an agent or compound which can inhibit or partially inhibits the activation of AT₁R. This includes antagonists for AT₁R, inverse agonists and negative allosteric modulators.

The term “AT₁R blocker” includes pharmaceutically acceptable salts of AT₁R blockers. Alternatively, the AT₁R blocker may be an antibody blocker of the AT₁R.

For example, the AT₁R blocker may be selected from the group comprising: irbesartan (e.g. Avapro®), eprosartan (e.g. Teveten®), losartan (e.g. Cozaar®), valsartan (e.g. Diovan®), telmisartan (e.g. Micardis®), candesartan (e.g. Atacand®), olmesartan (e.g. Benicar®), azilsartan (e.g. Edarbi®) and ZD-7115. As an example, the angiotensin receptor inhibitor may be irbesartan.

Both the CXCR2 inhibitor and the AT₁R blocker may be the same active agent, for example a bi-specific antibody.

Both the AT₁R blocker and the CXCR2 inhibitor may be pharmaceutically acceptable salts of the respective active agent. Pharmaceutically and veterinary acceptable salts include salts which retain the biological effectiveness and properties of the compounds of the present disclosure and which are not biologically or otherwise undesirable. In many cases, the compounds disclosed herein are capable of forming acid and/or base salts by virtue of the presence of amino and/or carboxyl groups or groups similar thereto. Acceptable base addition salts can be prepared from inorganic and organic bases. Salts derived from inorganic bases, include by way of example only, sodium, potassium, lithium, ammonium, calcium and magnesium salts. Salts derived from organic bases include, but are not limited to, salts of primary, secondary and tertiary amines, such as by way of example only, alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amines, trisubstituted cycloalkyl amines, cycloalkenyl amines, di(cycloalkenyl) amines, tri(cycloalkenyl) amines, substituted cycloalkenyl amines, disubstituted cycloalkenyl amines, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group.

Pharmaceutically and veterinary acceptable acid addition salts may be prepared from inorganic and organic acids. The inorganic acids that can be used include, by way of example only, hydrochloric acid, hydrobromic acid, sulfuric acid, nitric acid, phosphoric acid, and the like. The organic acids that can be used include, by way of example only, acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The pharmaceutically or veterinary acceptable salts of the compounds useful in the present disclosure can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences. 17th ed., Mack Publishing Company, Easton, Pa. (1985), p. 1418, the disclosure of which is hereby incorporated by reference. Examples of such acceptable salts are the iodide, acetate, phenyl acetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, γ-hydroxybutyrate, β-hydroxybutyrate, butyne-1,4-dioate, hexyne-1,4-dioate, hexyne-1,6-dioate, caproate, caprylate, chloride, cinnamate, citrate, decanoate, formate, fumarate, glycollate, heptanoate, hippurate, lactate, malate, maleate, hydroxymaleate, malonate, mandelate, mesylate, nicotinate, isonicotinate, nitrate, oxalate, phthalate, terephthalate, phosphate, monohydrogenphosphate, dihydrogenphosphate, metaphosphate, pyrophosphate, propiolate, propionate, phenylpropionate, salicylate, sebacate, succinate, suberate, sulfate, bisulfate, pyrosulfate, sulfite, bisulfite, sulfonate, benzenesulfonate, p-bromophenylsulfonate, chlorobenzenesulfonate, propanesulfonate, ethanesulfonate, 2-hydroxyethanesulfonate, merhanesulfonate, naphthalene-1-sulfonate, naphthalene-2-sulfonate, p-toluenesulfonate, xylenesulfonate, tartarate, and the like.

### Condition or Disease

Preferably the CXCR2 pathway inhibitor and/or the AT₁R blocker are associated with a condition or disease. More preferably, the condition or disease is Chronic Obstructive Pulmonary Disease (COPD). COPD is an umbrella term used to describe progressive lung diseases including emphysema, chronic bronchitis, bronchiectasis and refractory (non-reversible) asthma. These diseases are characterized by increasing breathlessness due to airflow limitations in the lungs.

### Measurement of Inhibition

The inhibition or partial inhibition of the CXCR2 pathway and/or the AT₁R caused by (i) the CXCR2 pathway inhibitor, (ii) the AT₁R blocker, or (iii) a combination of both the CXCR2 pathway inhibitor and the AT₁R blocker, may be measured using the in vitro methods set out herein, and include but are not limited to, biochemical or cellular assays for the assessment of in vitro chemotactic migration of CXCR2-expressing neutrophils and other cells such as are known in the art, as well as measurement of inositol phosphate production, extracellular-regulated kinase (ERK) phosphorylation, cAMP production, label-free technologies (such as using impedance, light refraction or charge redistribution), G protein coupling using proximity reporter systems or other approaches, β-arrestin recruitment or mediated signalling, transcription factor-based reporter systems, microscopy visualization using fluorescent labels, use of antibodies to assess receptor cellular localization (such as enzyme-linked immunosorbent assays), use of bioluminescence resonance energy transfer to assess cellular localization and trafficking, and fluorescence activated cell sorting.

The inhibition or partial inhibition of the CXCR2 pathway and/or the AT₁R caused by (i) the CXCR2 pathway inhibitor, (ii) the AT₁R blocker, or (iii) a combination of both the CXCR2 pathway inhibitor and the AT₁R blocker, may be measured using the in vivo methods set out herein, and include but are not limited to, measurement of cellular and cytokine content of lung exudate, measurement of lung function including physical capacity of lung function using spirometry-based tests, or lung functional outputs measured using measurement blood gas or other biochemical measures, or improvement in functional benefit including clinical benefit measured by quantitative methods such as walk tests or qualitative methods such as patient-reported outcome assessment. Inhibition or partial inhibition may be indicated by a qualitative improvement in lung structure as measured by one or more of the above-mentioned endpoints.

In one preferred embodiment, the total efficacy of the pharmaceutical formulation is greater when compared to the efficacies of the AT₁R blocker or the CXCR2 pathway inhibitor when either component is administered without any administration of the other component. Thus, the combined formulation may be administered in a single dose, including at subtherapeutic doses, or less often, than either of the two components might be administered as single compounds.

Preferably, the total efficacy of the pharmaceutical formulation is greater when compared to the sum of the efficacies of the AT₁R blocker and the CXCR2 pathway inhibitor when either component is administered without any administration of the other component. More preferably, a synergistic effect in efficacy is observed when the AT₁R blocker and the CXCR2 pathway inhibitor are administered concurrently or sequentially.

Alternatively, the total efficacy of the pharmaceutical formulation is equal to the sum of the efficacies of the AT₁R blocker and the CXCR2 pathway inhibitor when either component is administered without any administration of the other component. As a further preferred embodiment of this alternative, an additive effect in efficacy is observed when the AT₁R blocker and the CXCR2 pathway inhibitor are administered concurrently or sequentially.

In a further alternative, the total efficacy of the pharmaceutical formulation is less than the sum of the efficacies of the AT₁R blocker and the CXCR2 pathway inhibitor when either component is administered without any administration of the other component. In a further embodiment, while the combined efficacy is less than the sum of the efficacies of the AT₁R blocker and the CXCR2 pathway inhibitor when each component is administered without any administration of the other component, the treatment provides greater efficacy compared to a single treatment of AT₁R blocker or the CXCR2 pathway inhibitor administered alone.

Preferably the two components are administered concurrently at the same time (for example as two tablets taken together, or as a single tablet, formulated with each component) or sequentially (for example one tablet taken after another tablet). The doses of each component may be taken together (concurrently), or sequentially and taken within seconds, minutes, days, weeks or months of each other.

One component of the combination of the present invention may already be being administered to a subject, for example as standard of care treatment. In such a case, the second component of the combination of the present invention is administered as a second component in therapy to provide the therapeutic combination of the present invention.

### Method of Treatment

The invention further provides a method for treatment, amelioration or prevention of a condition or disease, said method comprising the step of:
i) administering to a subject a therapeutically effective amount of a combination of (a) an angiotensin type 1 receptor (AT₁R) blocker, and (b) a CXC chemokine receptor 2 (CXCR2) pathway inhibitor.

The subject to be treated is preferably a mammal, including a human mammal.

Preferably the condition or disease that is to be treated, ameliorated or prevented is COPD. Preferably the COPD is selected from: emphysema, chronic bronchitis, bronchiectasis, and refractory (non-reversible) asthma.

The CXCR2 inhibitor and the AT₁R blocker may be administered: in the same dosage form or in separate dosage forms.

The CXCR2 inhibitor and/or the AT₁R blocker may be antibody inhibitors or blockers of the respective receptors. The CXCR2 inhibitor and the AT₁R blocker may be the same active agent, for example a bi-specific antibody. The CXCR2 inhibitor and/or the AT₁R blocker may be pharmaceutically acceptable salts of the CXCR2 inhibitor and/or the AT₁R blocker.

The CXCR2 inhibitor and the AT₁R blocker may be administered: concurrently or sequentially.

One component of the combination of the present invention may already be being administered to a subject, for example as standard of care treatment. In such a case, the second component of the combination of the present invention is administered as a second component in therapy to provide the therapeutic combination of the present invention.

While not intending to be restricted to any particular mode of action, in one preferred embodiment the CXCR2 inhibitor has a greater affinity and/or potency and/or efficacy when interacting with the CXCR2 or modulating its downstream pathways when the CXCR2 is associated with the angiotensin receptor AT₁R. For example, the CXCR2 and the angiotensin receptor AT₁R may be associated as a CXCR2/AT₁R heteromer.

Preferably, the combination of the present invention provides a lower dose of the AT₁R or CXCR2 inhibitor than when either one is used alone. For example, one or both of the AT₁R or CXCR2 inhibitor may be provided at a subtherapeutic dose. This would have the benefit of reducing the negative safety profile of the AT1R or CXCR2 inhibitor while having the same therapeutic benefit for COPD.

In a further preferred embodiment, when the CXCR2 inhibitor is administered to a subject concurrently or sequentially with an AT1R blocker, the combined affinity, potency and/or efficacy is greater than compared to the affinity, potency and/or efficacy that would have been achieved when the CXCR2 inhibitor is not administered in combination (whether concurrently or sequentially) with the AT1R blocker. In an even further preferred embodiment, a synergistic effect (as measured by affinity, potency and/or efficacy) is achieved when the CXCR2 inhibitor is administered to a subject in combination (whether concurrently or sequentially) with an AT₁R blocker.

While not intending to be restricted to any particular mode of action, in one preferred embodiment the AT₁R blocker has a greater affinity and/or potency and/or efficacy when interacting with the angiotensin receptor AT₁R when the angiotensin receptor AT₁R is associated with the CXCR2. For example, the CXCR2 and the angiotensin receptor AT₁R may be associated as a CXCR2/AT₁R heteromer. In a further preferred embodiment, when the AT₁R blocker is administered to a subject concurrently or sequentially with a CXCR2 inhibitor, the combined affinity, potency and/or efficacy is greater than compared to the affinity, potency and/or efficacy that would have been achieved when the AT₁R blocker is not administered in combination (whether concurrently or sequentially) with the CXCR2 inhibitor. In an even further preferred embodiment, a synergistic effect (as measured by affinity, potency and/or efficacy) is achieved when the AT₁R blocker is administered to a subject in combination (whether concurrently or sequentially) with a CXCR2 inhibitor.

### Delivery

The dosage form provided by the present invention may further comprise a vial, cartridge, container, tablet or capsule comprising the pharmaceutical formulation of the invention together with dosage instructions for the administration of the dosage form to a subject for the treatment, amelioration or prevention of a condition or disease.

The amount of each active ingredient which may be combined with the carrier materials to produce a single dosage will vary, depending upon the host to be treated and the particular mode of administration. For example, a formulation intended for oral administration to humans may contain about 0.5 mg to 1 g of each active compound with an appropriate and convenient amount of carrier material, which may vary from about 5 to 95 percent of the total formulation. Dosage unit forms will generally contain between from about 0.5 mg to 500 mg of active ingredient(s).

Preferably, the AT₁R blocker is provided at between 50 mg to 500 mg per day, provided in one or more doses. Even more preferably, the AT₁R blocker is provided at between 75 mg to 300 mg per day. For example, the AT₁R blocker is irbesartan and is administered at a dose of 75, 150 or 300 mg per day, provided in one or more doses.

Preferably, the CXCR2 pathway inhibitor is provided at between 0.5 mg to 2000 mg per day, provided in one or more doses. Even more preferably the CXCR2 pathway inhibitor is provided at a dose of between 0.5 mg to 50 mg per day, provided in one or more doses.

The dose of each active agent may be provided in either a single dosage form, or two separate dosage forms and may comprise about 5 mg to 1 g of the AT₁R blocker, and about 0.5 mg to 1 g of the CXCR2 pathway inhibitor. The dose of the two actives may be provided in either a single dosage form, or two separate dosage forms and may comprise (i) a daily dose of AT₁R blocker of between about 50 mg to 500 mg, and (ii) a daily dose of CXCR2 pathway inhibitor of between about 5 mg to 50 mg. The AT₁R blocker may be irbesartan, and the dosage form may comprise a daily dose of irbesartan of about 300 mg.

It will be understood, however, that the specific dose level for any particular subject will depend upon a variety of factors including the activity of the specific compound employed, the age, body weight, general health, sex, diet, time of administration, route of administration, rate of excretion, drug combination and the severity of the particular condition or disease undergoing therapy.

Formulations of the invention, in various aspects, may be administered by injection, or prepared for oral, pulmonary, nasal or for any other form of administration. Preferably the formulations are administered, for example, intravenously, subcutaneously, intramuscularly, intraorbitally, ophthalmically, intraventricularly, intracranially, intracapsularly, intraspinally, intracisternally, intraperitoneally, buccal, rectally, vaginally, intranasally or by aerosol administration.

The mode of administration is in one aspect at least suitable for the form in which the formulation has been prepared. The mode of administration for the most effective response may be determined empirically and the means of administration described below are given as examples, and do not limit the method of delivery of the formulation of the present invention in any way. All the formulations provided are commonly used in the pharmaceutical industry and are commonly known to suitably qualified practitioners.

### Injectable Dosage Forms

The formulations of the invention in certain aspects may include pharmaceutically acceptable non-toxic excipients and carriers and administered by any parenteral techniques such as subcutaneous, intravenous and intraperitoneal injections. In addition, the formulations may optionally contain one or more adjuvants. As used herein, a “pharmaceutical carrier” is a pharmaceutically acceptable solvent, suspending agent, excipient or vehicle for delivering the compounds to the subject. The carrier may be liquid or solid and is selected with the planned manner of administration in mind.

The pharmaceutical forms suitable for injectable use optionally include sterile aqueous solutions (where water-soluble) or dispersions and sterile powders for the extemporaneous preparation of sterile injectable solutions or dispersion. Alternatively, the compounds of the invention are, in certain aspects encapsulated in liposomes and delivered in injectable solutions to assist their transport across cell membrane. Alternatively, or in addition, such preparations contain constituents of self-assembling pore structures to facilitate transport across the cellular membrane. The carrier, in various aspects, is a solvent or dispersion medium containing, for example, water, ethanol, polyol (for example, glycerol, propylene glycol and liquid polyethylene glycol, and the like), suitable mixtures thereof, and vegetable oils. Proper fluidity is maintained, for example and without limitation, by the use of a coating such as lecithin, by the maintenance of the required particle size in the case of dispersion and by the use of surfactants. Prolonged absorption of the injectable formulations is in certain aspects brought about by the use in the formulations of agents delaying absorption, for example, aluminium mono-stearate and gelatine.

The invention also provides an injectable sustained release pharmaceutical formulation comprising a therapeutically effective pharmaceutical formulation according to the invention, and a release retardant. The release retardant may be, for example, aluminium mono-stearate and gelatine.

Sterile injectable solutions are prepared by incorporating the active compounds in the required amount in an appropriate solvent with one or more of the other ingredients enumerated above, as required, followed by filtered sterilisation. Generally, dispersions are prepared by incorporating the various sterilised active ingredient into a sterile vehicle that contains the basic dispersion medium and the required other ingredients from those enumerated above. In the case of sterile powders for the preparation of sterile injectable solutions, preparation in certain aspects include without limitation vacuum drying and freeze-drying techniques that yield a powder of the active ingredient plus any additional desired ingredient from previously sterile-filtered solution thereof.

### Oral Dosage Forms

Contemplated for use herein are oral solid dosage forms, which are described generally in Martin, Remington's Pharmaceutical Sciences, 18th Ed. (1990 Mack Publishing Co. Easton PA 18042) at Chapter 89, which is herein incorporated by reference. Solid dosage forms include tablets, capsules, pills, troches or lozenges, cachets or pellets. Also, liposomal or proteinoid encapsulation may be used to formulate the present formulations (as, for example, proteinoid microspheres reported in U.S. Pat. No. 4,925,673). Liposomal encapsulation may be used and the liposomes may be derivatised with various polymers (E.g., U.S. Pat. No. 5,013,556). A description of possible solid dosage forms for the therapeutic is given by Marshall, in Modern Pharmaceutics, Chapter 10, Banker and Rhodes ed., (1979), herein incorporated by reference. In general, the formulation will include the compounds described as part of the invention (or a chemically modified form thereof), and inert ingredients which allow for protection against the stomach environment, and release of the biologically active material in the intestine.

For the CXCR2 pathway inhibitor or AT₁R blocker of the invention the location of release may be the stomach, the small intestine (the duodenum, the jejunum, or the ileum), or the large intestine. One skilled in the art has available formulations that will not dissolve in the stomach yet will release the material in the duodenum or elsewhere in the intestine. In one aspect, the release will avoid the deleterious effects of the stomach environment, either by protection of the formulation or by release of the compounds beyond the stomach environment, such as in the intestine.

The invention further provides an oral sustained release pharmaceutical formulation comprising a therapeutically effective pharmaceutical formulation according to the invention, and a release retardant.

In one aspect of the present invention the release retardant is a water-soluble, water swellable and/or water insoluble polymer. In particular, water-soluble polymers are selected from the group comprising are ethylcellulose, hydroxypropyl cellulose, hydroxypropyl methyl cellulose, an enteric coating; and a semipermeable membrane. In another aspect of the invention the release retardant is a non-polymeric release retardant. More particularly, the non-polymeric release retardant is hydrogenated castor oil. The formulations of the invention may be milled or granulated and compressed into tablets or encapsulated into capsules according to conventional procedures known in the art.

To ensure full gastric resistance, a coating impermeable to at least pH 5.0 is used. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

A coating or mixture of coatings can also be used on tablets, which are not intended for protection against the stomach. This includes without limitation sugar coatings, or coatings that make the tablet easier to swallow. Exemplary capsules consist of a hard shell (such as gelatine) for delivery of dry therapeutic i.e. powder; for liquid forms, a soft gelatine shell may be used. The shell material of cachets in certain aspects is thick starch or other edible paper. For pills, lozenges, moulded tablets or tablet triturates, moist massing techniques are also contemplated, without limitation.

As used herein, the term “sustained release” means the gradual but continuous or sustained release over a relatively extended period of the therapeutic compound content after oral ingestion. The release may continue after the pharmaceutical formulation has passed from the stomach and through until and after the pharmaceutical formulation reaches the intestine. The phrase “sustained release” also means delayed release wherein release of the therapeutic compound is not immediately initiated upon the pharmaceutical formulation reaching the stomach but rather is delayed for a period of time, for example, until when the pharmaceutical formulation reaches the intestine. Upon reaching the intestine, the increase in pH may then trigger release of the therapeutic compound from the pharmaceutical formulation.

Though term “release retardant” is used herein, means a substance that reduces the rate of release of a therapeutic compound from a pharmaceutical formulation when orally ingested. The release retardant may be a polymer or a non-polymer. The release retardant may be used according to any one of several sustained release systems including, for example, a diffusion system, a dissolution system and/or an osmotic system.

In certain aspects, the therapeutic is included in the formulation as fine multiparticulates in the form of granules or pellets of particle size about 1 mm. The formulation of the material for capsule administration is, in certain aspects, a powder, lightly compressed plugs or even as tablets. In one aspect, the therapeutic could be prepared by compression.

Colourants and flavouring agents may optionally be included. For example, compounds may be formulated (such as, and without limitation, by liposome or microsphere encapsulation) and then further contained within an edible product, such as a refrigerated beverage containing colorants and flavouring agents.

The volume of the therapeutics may, in one aspect, be diluted or increased with an inert material. These diluents could include carbohydrates, especially mannitol, alpha-lactose, anhydrous lactose, cellulose, sucrose, modified dextrans and starch. Certain inorganic salts are also optionally used as fillers including calcium triphosphate, magnesium carbonate and sodium chloride. Some commercially available diluents are Fast-Flo, Emdex, STA-Rx 1500, Emcompress and Avicell.

In other embodiments, disintegrants are included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatine, orange peel, acid carboxymethyl cellulose, natural sponge and bentonite are also contemplated. Another form of the disintegrants is the insoluble cationic exchange resins. Powdered gums are also optionally used as disintegrants and as binders and these include, without limitation, powdered gums such as agar, Karaya or tragacanth. Alginic acid and its sodium salt are also useful as disintegrants.

Binders are contemplated to hold the therapeutic compounds together to form a hard tablet and include, without limitation, materials from natural products such as acacia, tragacanth, starch and gelatine. Other binders include, without limitation, methylcellulose (MC), ethyl cellulose (EC) and carboxymethyl cellulose (CMC). Polyvinyl pyrrolidone (PVP) and hydroxypropylmethyl cellulose (HPMC) are contemplated for use in alcoholic solutions to granulate the therapeutic.

An antifrictional agent may be optionally included in the formulation of the therapeutic to prevent sticking during the formulation process. Lubricants may be optionally used as a layer between the therapeutic and the die wall, and these can include but are not limited to: stearic acid including its magnesium and calcium salts, polytetrafluoroethylene (PTFE), liquid paraffin, vegetable oils and waxes. Exemplary soluble lubricants may also be used such as include sodium lauryl sulfate, magnesium lauryl sulfate, polyethylene glycol of various molecular weights, and Carbowax 4000 and 6000.

Glidants that might improve the flow properties of the compound during formulation and to aid rearrangement during compression might be optionally added. The glidants may include without limitation starch, talc, pyrogenic silica and hydrated silicoaluminate.

To aid dissolution of the therapeutic into the aqueous environment, a surfactant might be added in certain embodiments as a wetting agent. Surfactants may include, for example and without limitation, anionic detergents such as sodium lauryl sulfate, dioctyl sodium sulfosuccinate and dioctyl sodium sulfonate. Cationic detergents might be optionally used and could include, without limitation, benzalkonium chloride or benzethomium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. When used, these surfactants could be present in the formulation of the compounds either alone or as a mixture in different ratios.

Additives that potentially enhance uptake of the compounds may be desirable. Such additives include the fatty acids oleic acid, linoleic acid and linolenic acid.

Controlled release formulation may be desirable. In certain aspects, the compounds could be incorporated into an inert matrix that permits release by either diffusion or leaching mechanisms i.e., gums. In some aspects, slowly degenerating matrices may also be incorporated into the formulation. Another form of a controlled release of this therapeutic is by a method based on the OROS™ therapeutic system (Alza Corp.), i.e. the drug is enclosed in a semipermeable membrane which allows water to enter and push drug out through a single small opening due to osmotic effects. Some enteric coatings also have a delayed release effect.

In other aspects, a mix of materials might be used to provide the optimum film coating. Film coating may be carried out, for example and without limitation, in a pan coater or in a fluidized bed or by compression coating.

### Pulmonary and Nasal Dosage Forms

Also contemplated herein is pulmonary delivery of the formulations of the invention. In these aspects, the AT₁R blocker or the CXCR2 pathway inhibitor may be delivered to the lungs of a subject while inhaling and traverses across the lung epithelial lining to the blood stream.

Contemplated for use in the practice of this invention are a wide range of mechanical devices designed for pulmonary delivery of therapeutic products, including but not limited to nebulizers, metered-dose inhalers, and powder inhalers, all of which are familiar to those skilled in the art.

Some specific examples of commercially available devices suitable for the practice of this invention are, for example and without limitation, the Ultravent™ nebulizer, manufactured by Mallinckrodt, Inc., St. Louis, Missouri; the Acorn™ II nebulizer, manufactured by Marquest Medical Products, Englewood, Colorado; the Ventolin™ metered dose inhaler, manufactured by Glaxo Inc., Research Triangle Park, North Carolina; and the Spinhaler™ powder inhaler, manufactured by Fisons Corp., Bedford, Massachusetts.

All such devices require the use of formulations suitable for the dispensing of the compounds. Typically, each formulation is specific to the type of device employed and may involve the use of an appropriate propellant material, in addition to the usual diluents, adjuvants and/or carriers useful in therapy. Also, the use of liposomes, microcapsules or microspheres, inclusion complexes, or other types of carriers is contemplated.

Formulations suitable for use with a nebulizer, either jet or ultrasonic, will typically comprise the compounds suspended in water. The formulation may also include, in one aspect, a buffer and a simple sugar (e.g., for protein stabilization and regulation of osmotic pressure). In one embodiment, the nebulizer formulation may also contain a surfactant, to reduce or prevent surface induced aggregation of the compounds caused by atomization of the solution in forming the aerosol.

Formulations for use with a metered dose inhaler device will generally comprise, in one aspect a finely divided powder containing the compounds suspended in a propellant with the aid of a surfactant. The propellant may be is any conventional material employed for this purpose, such as and without limitation, a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2 tetrafluoroethane, or combinations thereof. Suitable surfactants include, without limitation sorbitan trioleate and soya lecithin. Oleic acid may also be useful as a surfactant in certain aspects.

Formulations for dispensing from a powder inhaler device will comprise a finely divided dry powder containing the compound and may also include a bulking agent, such as and without limitation lactose, sorbitol, sucrose, or mannitol in amounts which facilitate dispersal of the powder from the device, e.g., 50 to 90% by weight of the formulation. In certain embodiments, the compound(s) is/are prepared in particulate form with an average particle size of less than 10 microns, most preferably 0.5 to 5 microns, for most effective delivery to the distal lung.

Nasal delivery of the compounds is also contemplated. Nasal delivery allows the passage of the protein to the blood stream directly after administering the therapeutic product to the nose, without the necessity for deposition of the product in the lung. Formulations for nasal delivery include those with, for example and without limitation, dextran or cyclodextran.

### Dosing Schedule

It will be appreciated that in certain aspects, the formulations of the invention may be given as a single dose schedule, or preferably, in a multiple dose schedule. A multiple dose schedule is one in which a primary course of delivery may be with 1 to 10 separate doses, is optionally followed by other doses given at subsequent time intervals required to maintain or reinforce the treatment. The dosage regimen will also, at least in part, be determined by the needs of the individual and the judgement of the practitioner.

The invention thus provides a tablet comprising the pharmaceutical formulation of the invention; a capsule comprising the pharmaceutical formulation of the invention, an injectable suspension comprising the pharmaceutical formulation of the invention, and a formulation for pulmonary delivery comprising the pharmaceutical formulation of the invention. The AT₁R blocker and the CXCR2 pathway inhibitor may be delivered in the same formulation, or may be delivered in separate formulations.

The AT₁R blocker and the CXCR2 pathway inhibitor may be in the same dosage form, or may be in separate dosage forms. The subject being administered the AT₁R blocker and the CXCR2 pathway inhibitor may be already receiving one of the active agents and may, in accordance with the present invention, be administered the other component of the treatment of the present invention.

### Use

The invention also provides for the use of a pharmaceutical formulation comprising (a) at least one angiotensin type 1 receptor (AT₁R) blocker, and (b) at least one CXC chemokine receptor 2 (CXCR2) pathway inhibitor for the manufacture of a formulation for the treatment, amelioration or prevention of a condition or disease.

The present invention further provides at least one AT₁R blocker, and at least one CXCR2 inhibitor, for use in a formulation for the treatment, amelioration or prevention of a disease.

The present invention further provides at least one AT₁R blocker for use in a formulation for the treatment, amelioration or prevention of a disease wherein the at least one AT₁R blocker is administered to the subject concurrently or sequentially with at least one CXCR2 inhibitor.

The present invention further provides at least one CXCR2 inhibitor for use in a formulation for the treatment, amelioration or prevention of a disease wherein the at least one CXCR2 inhibitor is administered to the subject concurrently or sequentially with at least one AT₁R blocker.

Preferably the formulation is for use in the treatment, amelioration or prevention of a condition or disease that is COPD. Preferably the COPD is selected from: emphysema, chronic bronchitis, bronchiectasis, and refractory (non-reversible) asthma.

The CXCR2 inhibitor and the AT₁R blocker may be administered: in the same dosage form or in separate dosage forms.

The CXCR2 inhibitor and/or the AT₁R blocker may be an antibody inhibitor or blocker of the respective receptors. The CXCR2 inhibitor and the AT₁R blocker may be the same active agent, for example a bi-specific antibody. The CXCR2 inhibitor and/or the AT₁R blocker may be pharmaceutically acceptable salts of the CXCR2 inhibitor and/or the AT₁R blocker.

The CXCR2 inhibitor and the AT₁R blocker may be administered: concurrently or sequentially.

### Kits

The present invention provides a kit for the treatment or prevention of a condition or disease, said kit comprising:
a) at least one angiotensin type 1 receptor (AT₁R) blocker;
b) at least one CXC chemokine receptor 2 (CXCR2) pathway inhibitor; and
c) instructions for use.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. For in vivo use, the expression construct may be formulated into a pharmaceutically acceptable syringeable composition. In this case the container means may itself be an inhalant, syringe, pipette, eye dropper, or other such like apparatus, from which the formulation may be applied to an affected area of the animal, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye dropper or any such medically approved delivery vehicle.

### General

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (eg. Size, displacement and field strength etc). A range of values will be understood to include all values within the range, including the values defining the range, and values adjacent to the range which lead to the same or substantially the same outcome as the values immediately adjacent to that value which defines the boundary to the range. Accordingly, unless indicated to the contrary, the numerical parameters set forth in the specification and claims are approximations that may vary depending upon the desired properties sought to be obtained by the present invention. Hence “about 80%” means “about 80%” and also “80%”. At the very least, each numerical parameter should be construed in light of the number of significant digits and ordinary rounding approaches.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs. The term “active agent” may mean one active agent or may encompass two or more active agents.

The following examples serve to more fully describe the manner of using the above-described invention, as well as to set forth the best modes contemplated for carrying out various aspects of the invention. It is understood that these methods in no way serve to limit the true scope of this invention, but rather are presented for illustrative purposes.

### EXAMPLES

Further features of the present invention are more fully described in the following non-limiting Examples. This description is included solely for the purposes of exemplifying the present invention. It should not be understood as a restriction on the broad description of the invention as set out above.

In each of the following examples 1, 3, 4 and 5 independently, the following general materials and methods apply, unless the context requires otherwise.

HEK293FT cells were seeded in 6-well plates at a density of approximately 700,000 cells/well and maintained at 37° C., 5% CO₂ in Complete Media (DMEM containing 0.3 mg/ml glutamine, 100 IU/ml penicillin and 100 μg/ml streptomycin (Gibco/Thermo Fisher)) supplemented with 10% fetal calf serum (FCS; Bovogen). Transient transfections were carried out 24 h after seeding using FuGene6™ (Promega) according to manufacturer instructions. 24 h post-transfection, cells were washed with PBS, detached using 0.05% trypsin/0.53 mM EDTA, resuspended in HEPES-buffered phenol red free Complete Media containing 5% FCS and added to a poly-L-lysine-coated white 96-well microplate (Greiner Bio-One). 48 h post-transfection, bioluminescence resonance energy transfer (BRET) assays were carried out following pre-incubation of cells with EnduRen™ (Promega) at a final concentration of 30 μM, at 37° C., 5% CO₂ for 2 h. BRET measurements were taken at 37° C. using the LUMlstar™ or CLARIOstar™ (BMG Labtech). Filtered light emissions were measured for 1 s in each of the ‘donor wavelength window’ (460-490 nm for LUMlstar™ or 410-490 nm for CLARIOstar™) and ‘acceptor wavelength window’ (520-550 nm for LUMlstar™ or 520-620 nm for CLARIOstar™).

For antagonist/inverse agonist assays, cells were pre-incubated with EnduRen for 1.5 hours, followed by the addition of vehicle or antagonists/inverse agonists (10 μM) for another 30 minutes. After then reading for 20 minutes, the agonists (C-X-C Motif Chemokine Ligand 8 [CXCL8] 10 nM and/or Angiotensin II [AngII] 100 nM) were added (therefore 50 minutes after initial vehicle or antagonist/inverse agonist addition) and the assay was measured for another 40 minutes.

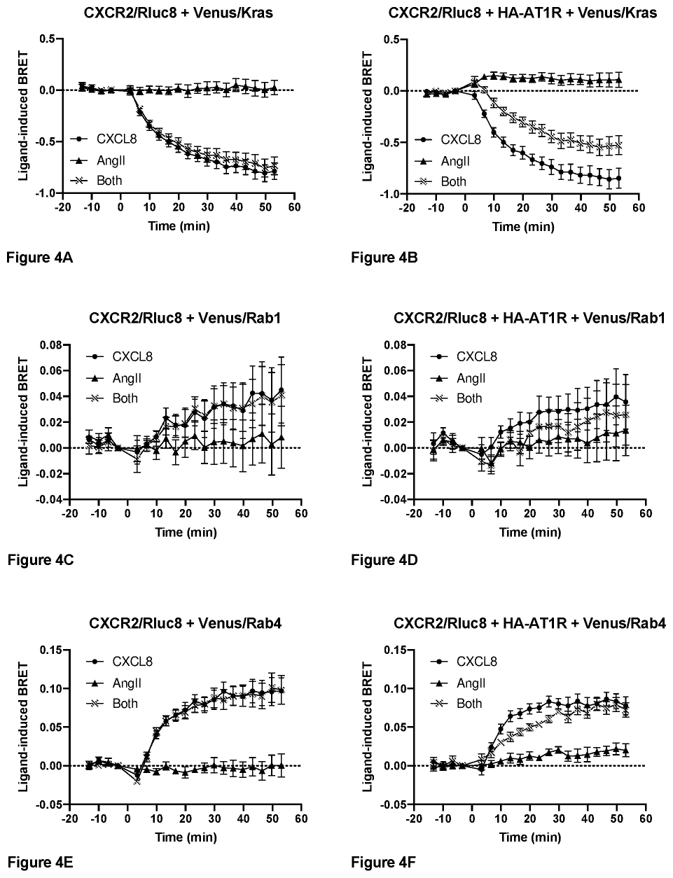

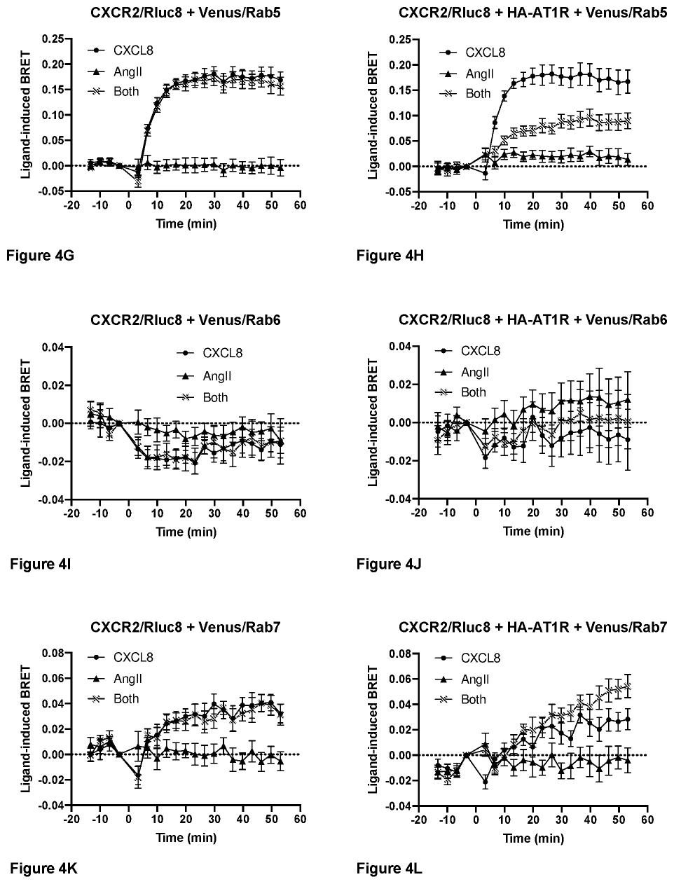

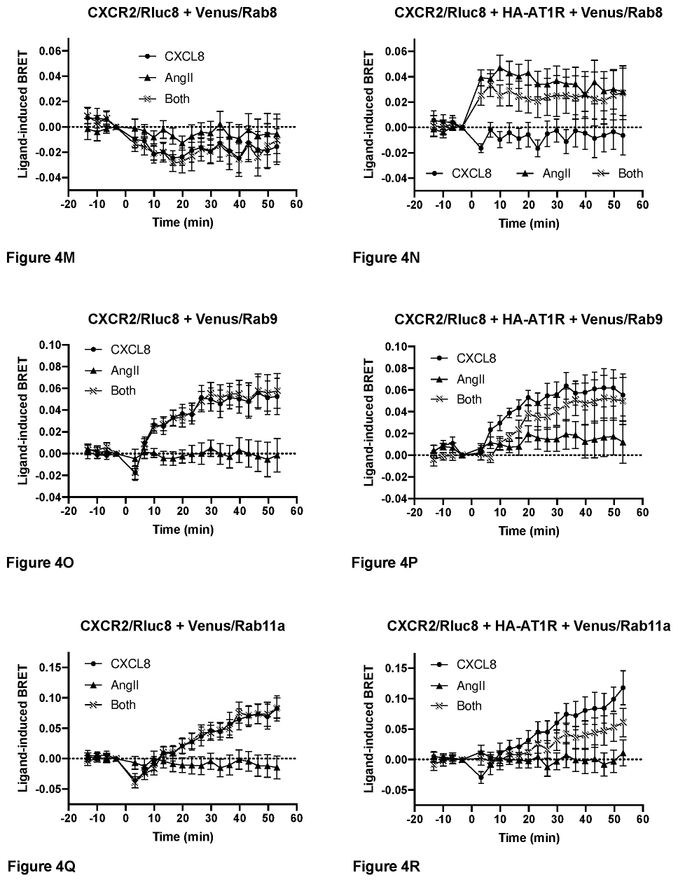

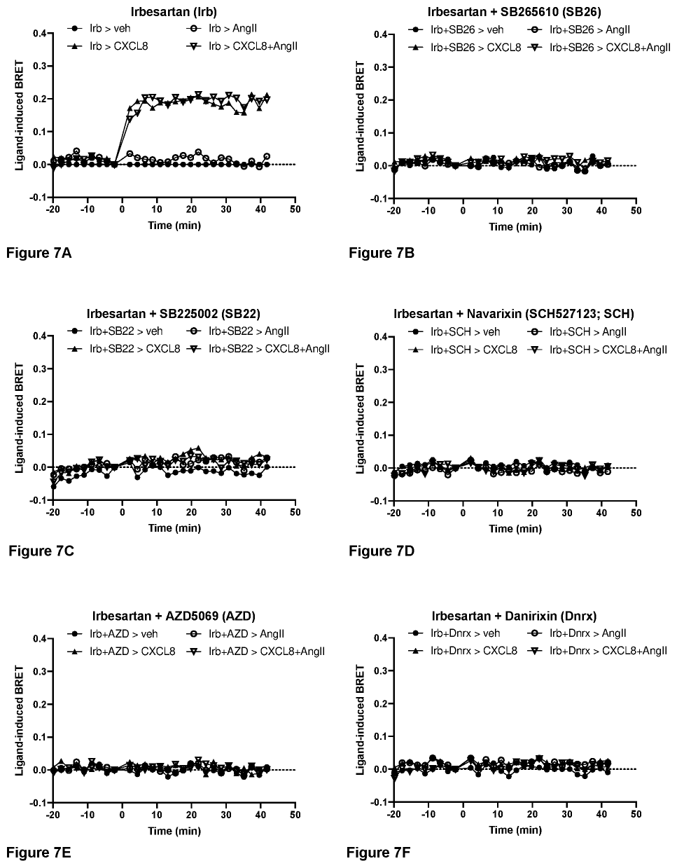

The BRET signal observed between interacting proteins is normalized by subtracting the background BRET ratio. This can be done in one of two ways (see Pfleger et al. (2006) Cell Signal 18:1664-1670; Pfleger et al. (2006) Nat Protoc 1:336-344): 1) the ratio of the 520-550 nm or 520-620 nm emission over the 460-490 nm or 410-490 nm emission for a cell sample containing only the donor construct is subtracted from the same ratio for a sample containing the interacting acceptor and donor fusion proteins; 2) the ratio of the 520-550 nm or 520-620 nm emission over the 460-490 nm or 410-490 nm emission for a cell sample treated with vehicle is subtracted from the same ratio for a second aliquot of the same cell sample treated with ligand. In the following examples, the second calculation will be used and the signal is described as ligand-induced BRET′. Furthermore, for data shown in FIGS. 4 to 20, all data points in a given set are corrected relative to the final data point prior to agonist treatment, which is fixed to zero. In keys on FIGS. 7 to 20, “>” means “followed by after 50 minutes”, with agonists added at time zero on the x-axes.

Mini G (mG) proteins are engineered GTPase domains of Ga subunits. They have been used in BRET assays fused to fluorescent proteins to report on G protein-coupled receptor (GPCR) activity. “Variants of mG proteins (mGs, mGsi, mGsq, and mG12) corresponding to the four families of Ga subunits displayed appropriate coupling to their cognate GPCRs, allowing quantitative profiling of subtype-specific coupling to individual receptors” (Wan et al. (2018) J Biol Chem. 293(19):7466-7473).

The Receptor-Heteromer Investigation Technology (Receptor-HIT) is an assay configuration that provides insights into receptor complexes (Ayoub et al. (2015) PLoS One 10(3):e0119803). It is also known as GPCR-HIT when assessing GPCRs. It is an assay configuration whereby one receptor (eg. CXCR2) is labelled with one component (eg. Renilla luciferase variant Rluc8) of a proximity-based reporter system (eg. BRET), the complementary component of which (eg. Venus yellow fluorescent protein) is fused to a receptor interacting partner (eg. mGsi). Treatment with a ligand (eg. AngII) selective for the receptor untagged with respect to the BRET assay (eg. hemagglutinin epitope-tagged AT1R; HA-AT1R) results in modulation of the proximity of the BRET-tagged receptor and the interacting partner, resulting in a change in BRET signal that is indicative of functional interaction between the two receptors.

### Example 1

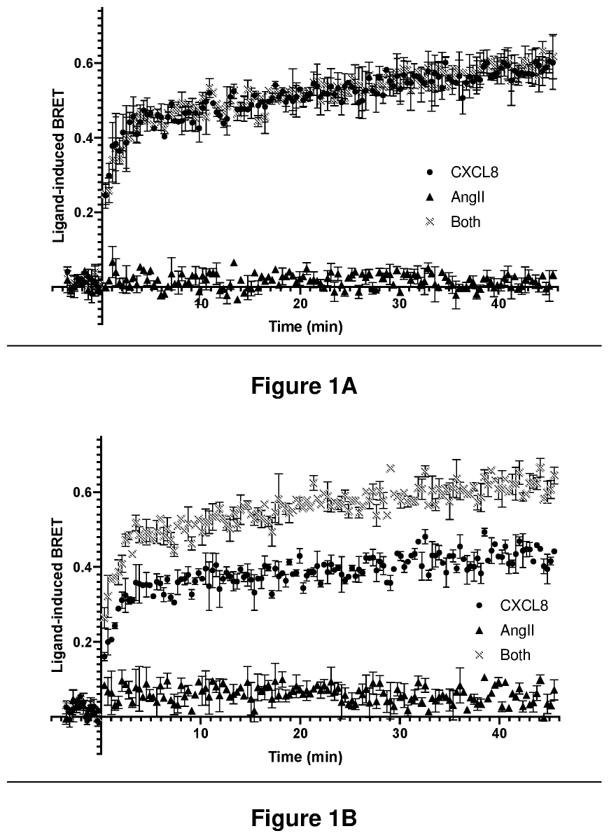

Referring now to FIG. 1, BRET signals were measured from cells transiently expressing CXCR2/Rluc8 (CXCR2 labelled with Rluc8) and Venus/mGsi (mGsi as a sensor for Gi activity labelled with Venus) with HA-AT1R following treatment with either 10⁻⁷M (100 nM) CXCL8 or 10⁻⁶M (1 μM) AngII only or both CXCL8 and AngII combined. In parallel, BRET signals were measured from cells transiently expressing CXCR2/Rluc8 and Venus/mGsi (no HA-AT1R expressed; pcDNA3 transfected instead to keep cDNA levels the same) following treatment with either 10⁻⁷M (100 nM) CXCL8 or 10⁻⁶M (1 μM) AngII only or both CXCL8 and AngII combined.

Prior to ligand treatment (added at 0 minutes), a baseline BRET signal was recorded for each of the combinations. AngII treatment of cells expressing CXCR2/Rluc8 and Venus/mGsi without HA-AT₁R did not result in an increase in ligand-induced BRET (FIG. 1A). CXCL8 treatment of cells expressing CXCR2/Rluc8 and Venus/mGsi without HA-AT₁R resulted in the BRET signal reaching about 0.6. A similar BRET signal was observed with combined treatment with AngII and CXCL8 in these cells.

In contrast, a discernible ligand-induced BRET signal was observed following AngII treatment of cells co-expressing CXCR2/Rluc8, Venus/mGsi and HA-AT₁R (FIG. 1B). This signal was approximately 0.05. Treatment of cells co-expressing CXCR2/Rluc8, Venus/mGsi and HA-AT₁R with CXCL8 resulted in a signal of approximately 0.4. Treatment of cells co-expressing CXCR2/Rluc8, Venus/mGsi and HA-AT₁R with both AngII and CXCL8 resulted in a signal of approximately 0.6, substantially greater than that observed following addition of CXCL8 or AngII alone.

This example demonstrates the greater than additive effect of combined treatment with CXCL8 and AngII, indicating that AngII activation of AT₁R has a synergistic effect on promoting Gi coupling to CXCL8-activated CXCR2.

### Example 2

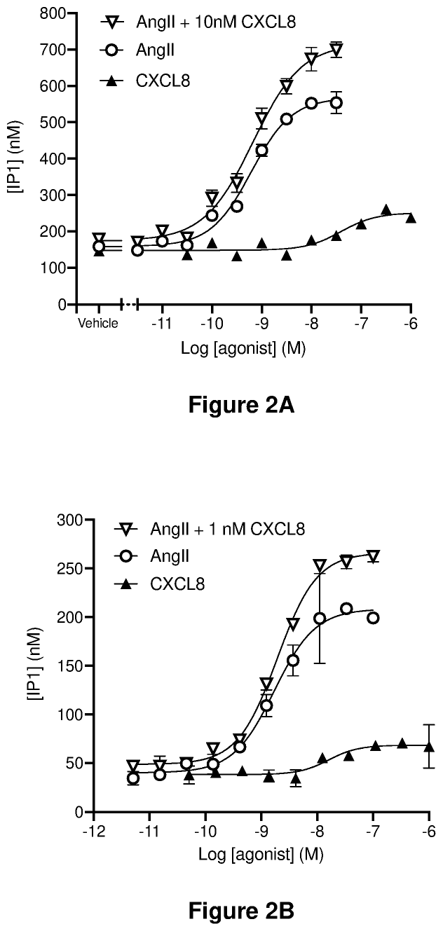

Referring now to FIG. 2, Receptor activation of the phospholipase C pathway was detected through the measurement of D-myo-Inositol 1-Phosphate (IP1) by competitive immunoassay (IP-one—Gq Kit, Cisbio 62IPAPEC). Human Embryonic Kidney (HEK) 293 cells were transiently transfected in suspension with the AGTR1 vector (Origene RC212008) alone or co-transfected with the CXCR2 vector (Origene Cat RC207794) using FuGene™ HD reagent (Promega E2311). 24 h later, experiments were performed in Stimulation buffer (Cisbio, 62IPAPEC)+0.1% Bovine Serum Albumin (BSA), dispensing 20,000 cells/well (7 μl) into a 384 well white Optiplate™ (Perkin Elmer 6007299), with 3.5 μl vehicle or Angiotensin II (AngII, Tocris 1158), and 3.5 μl vehicle or interleukin 8 (IL8, CXCL8, amino acids 28-99, R&D systems 208-IL-050) at the required final assay concentrations. Agonists were prepared in a threefold serial dilution in stimulation buffer+0.1% BSA and added to the assay to give the appropriate final concentration-response curves, and co-stimulation at a single concentration as required. An IP1 standard curve was also constructed in 14 μl Stimulation Buffer+0.1% BSA. Incubations were conducted for 90 min at 37° C., 5% CO₂, after which 3 μl IP1-d2 Reagent and 3 μl IP1 Tb Cryptate Antibody (Cisbio 62IPAPEC), in Lysis & Detection buffer, were added to each well for 60 min at room temperature. Plates were read on a HTRF® compatible reader (PHERAstar™, BMG Labtech), using 337 nm excitation and collecting donor/acceptor emission at 620 nm and 665 nm. A standard curve was plotted to interpolate unknown HTRF ratio (665/620×10 000) measurements as IP1 concentration (nM). The agonist concentration-response data were then fitted with a 4 parameter sigmoidal curve to derive log EC₅₀, and maximal response Rₘₐₓ where appropriate.

Accumulation of D-myo-Inositol 1-Phosphate (IP1) is known to occur as a consequence of receptors activating Gq proteins, in turn resulting in the activation of phospholipase C. AT1R is well known to activate the Gq signalling pathway (Cabana et al (2015) J Biol Chem 290, 15835-15854). It is also well established that activation of Gi protein (which is pertussis toxin-sensitive) can also activate certain isoforms of phospholipase C via the Gβγ subunits (Camps et al (1992) Nature 360, 684-686; Katz et al (1992) Nature 360, 686-689). CXCR2 classically signals via activation of Gi protein (Liu et al (2020) Nature 585, 135-140).

Referring now to FIG. 2A, HEK293 cells transiently-transfected with AT1R and CXCR2 treated with CXCL8 only resulted in a concentration-response curve with logEC₅₀ (M) of −7.40 and Rₘₐₓ of 251 nM. Treatment with AngII only resulted in a concentration-response curve with logEC₅₀ (M) of −9.21 and Rₘₐₓ of 567 nM. Treatment with 10 nM CXCL8 and AngII at a range of concentrations resulted in a concentration-response curve with logEC₅₀ (M) of −9.18 and Rₘₐₓ of 717 nM. It is notable that 10 nM CXCL8 in the absence of AngII results in [IP1] of about 171 nM, which is only about 12 nM higher than baseline (cells treated only with vehicle; 159 nM).

Referring now to FIG. 2B, HEK293 cells stably-expressing CXCR2 and transiently-transfected with AT1R treated with CXCL8 only resulted in a concentration-response curve with logEC₅₀ (M) of −7.79 and Rₘₐₓ of 68 nM. Treatment with AngII only resulted in a concentration-response curve with logEC₅₀ (M) of −8.77 and Rₘₐₓ of 209 nM. Treatment with 1 nM CXCL8 and AngII at a range of concentrations resulted in a concentration-response curve with logEC₅₀ (M) of −8.72 and Rₘₐₓ of 266 nM. It is notable that 1 nM CXCL8 in the absence of AngII results in [IP1] not discernibly different to baseline.

This example demonstrates that there is substantial potentiation of the [IP1], and therefore phospholipase C activity as a result of activating both AT1R with AngII and CXCR2 with CXCL8. Furthermore, this potentiation is consistent with the data showing a more than additive effect of treatment with both CXCL8 and AngII on Gi coupling (as demonstrated by proximity to the biosensor mGsi) when both AT1R and CXCR2 are present.

### Example 3

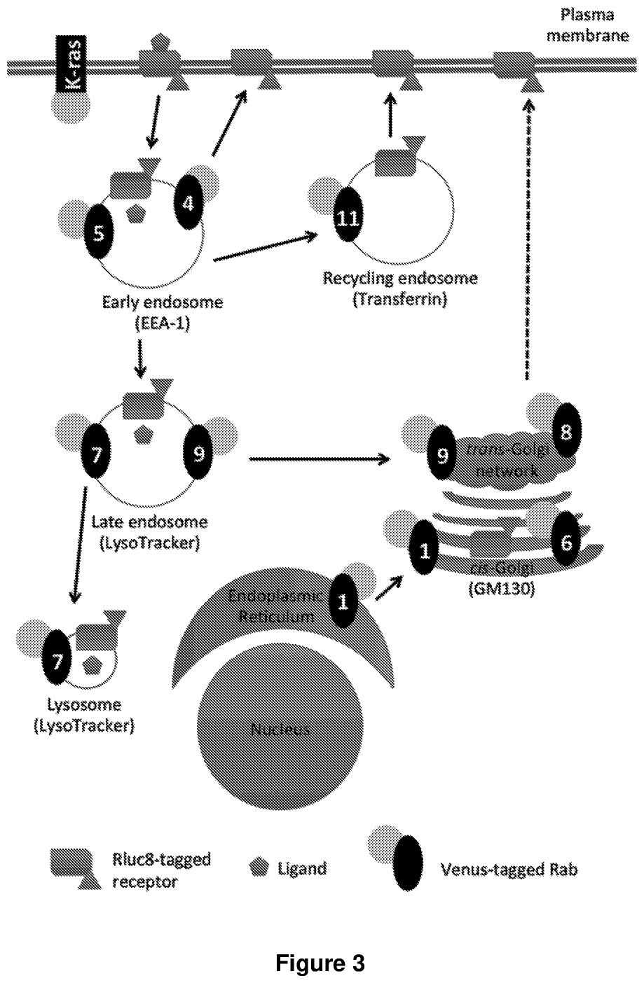

Referring now to FIG. 3 (published in Tiulpakov et al (2016) Mol. Endocrinol. 30, 889-904), the proximity of Rluc8-tagged receptors to Venus-tagged Kras plasma membrane marker, or Venus-tagged Rabs specific for distinct subcellular locations, is monitored in live cells and in real-time using BRET. The Rab markers are: Venus/Rab5 (5) for early endosomes; Venus/Rab4 (4) for early endosome recycling; Venus/Rab11a (11) for recycling endosomes; Venus/Rab7 (7) for late endosomes/lysosomes; Venus/Rab9 (9) for late endosome trafficking to the trans-Golgi network; Venus/Rab1 (1) for endoplasmic reticulum trafficking to the cis-Golgi; Venus/Rab6 (6) for Golgi apparatus and trans-Golgi network; or Venus/Rab8 (8) for trans-Golgi network to plasma membrane.

Referring now to FIG. 4A, in the absence of AT1R, CXCL8 treatment results in a marked reduction in the proximity of CXCR2/Rluc8 to plasma membrane marker Venus/Kras, indicating that CXCR2 is internalized into the cell. AngII has no effect and both CXCL8 and AngII combined has the same effect as CXCL8 only.

Referring now to FIG. 4B, in the presence of AT1R, CXCL8 treatment again results in a marked reduction in the proximity of CXCR2/Rluc8 to plasma membrane marker Venus/Kras, again indicating that CXCR2 is internalized into the cell. However, in contrast, AngII treatment results in increased proximity of CXCR2/Rluc8 and Venus/Kras, providing evidence for activation of the AT1R by AngII increasing the amount of CXCR2 on the plasma membrane. Without wishing to be bound by theory, these data indicate that either CXCR2 exhibits significant constitutive internalization that is inhibited by AngII activation of AT1R, and/or that activation of AT1R by AngII increases the forward trafficking of CXCR2 to the plasma membrane to increase the expression of the CXCR2 on the plasma membrane. Treatment with both CXCL8 and AngII resulted in less CXCR2 net internalization compared with CXCL8 only, which may be the net effect of internalization induced by CXCL8 and forward trafficking of CXCR2 to the plasma membrane induced by AT1R activation by AngII.

Referring now to FIG. 4C, in the absence of AT1R, CXCL8 treatment results in a small increase in the proximity of CXCR2/Rluc8 to Venus/Rab1, indicating a small increase in CXCR2 trafficking from the endoplasmic reticulum to the cis-Golgi. AngII has no effect and both CXCL8 and AngII combined has the same effect as CXCL8 only.

Referring now to FIG. 4D, in the presence of AT1R, CXCL8 treatment again results in a small increase in the proximity of CXCR2/Rluc8 to Venus/Rab1, again indicating a small increase in CXCR2 trafficking from the endoplasmic reticulum to the cis-Golgi. There is barely any effect of AngII only, however, co-treatment with AngII appears to reduce the effect of CXCL8.

Referring now to FIG. 4E, in the absence of AT1R, CXCL8 treatment results in an increase in the proximity of CXCR2/Rluc8 to Venus/Rab4, indicating an increase in early endosome recycling of CXCR2. AngII has no effect and both CXCL8 and AngII combined has the same effect as CXCL8 only.

Referring now to FIG. 4F, in the presence of AT1R, CXCL8 treatment again results in an increase in the proximity of CXCR2/Rluc8 to Venus/Rab4, indicating an increase in early endosome recycling of CXCR2. AngII only results in a very small increase in early endosome recycling of CXCR2 at later time-points, however, co-treatment with AngII appears to reduce the effect of CXCL8, particularly at earlier time-points.

Referring now to FIG. 4G, in the absence of AT1R, CXCL8 treatment results in a substantial increase in the proximity of CXCR2/Rluc8 to Venus/Rab5, indicating a substantial increase in CXCR2 in early endosomes. AngII has no effect and both CXCL8 and AngII combined has the same effect as CXCL8 only.

Referring now to FIG. 4H, in the presence of AT1R, CXCL8 treatment again results in a substantial increase in the proximity of CXCR2/Rluc8 to Venus/Rab5, indicating a substantial increase in CXCR2 in early endosomes. AngII only results in a very small increase in CXCR2 in early endosomes, however, co-treatment with AngII appears to substantially reduce the effect of CXCL8. This is consistent with reduced CXCL8-induced internalization of CXCR2 in the presence of AT1R activated by AngII, resulting in increased CXCR2 on the plasma membrane as observed with the Kras data.

Referring now to FIG. 4I, in the absence of AT1R, CXCL8 treatment results in a small decrease in the proximity of CXCR2/Rluc8 to Venus/Rab6, indicating a small decrease in CXCR2 trafficking through the Golgi apparatus to the trans-Golgi network. AngII has no effect and both CXCL8 and AngII combined has the same effect as CXCL8 only.

Referring now to FIG. 4J, in the presence of AT1R, CXCL8 treatment again results in a small decrease in the proximity of CXCR2/Rluc8 to Venus/Rab6, again indicating a small decrease in CXCR2 trafficking through the Golgi apparatus to the trans-Golgi network. In contrast, AngII only treatment results in a small increase in the proximity of CXCR2/Rluc8 to Venus/Rab6, indicating the AT1R activation by AngII promotes a small increase in CXCR2 trafficking through the Golgi apparatus to the trans-Golgi network. Co-treatment with AngII therefore appears to nullify the effect of CXCL8 at later time-points and vice versa.

Referring now to FIG. 4K, in the absence of AT1R, CXCL8 treatment results in a small increase in the proximity of CXCR2/Rluc8 to Venus/Rab7, indicating a small increase in CXCR2 in late endosomes/lysosomes. AngII has no effect and both CXCL8 and AngII combined has the same effect as CXCL8 only.

Referring now to FIG. 4L, in the presence of AT1R, CXCL8 treatment again results in a small increase in the proximity of CXCR2/Rluc8 to Venus/Rab7, again indicating a small increase in CXCR2 in late endosomes/lysosomes. There is barely any effect of AngII only, however, treatment with both AngII and CXCL8 results in a greater increase in proximity than observed with CXCL8 only, particularly at later time-points.

Referring now to FIG. 4M, in the absence of AT1R, CXCL8 treatment results in a small decrease in the proximity of CXCR2/Rluc8 to Venus/Rab8, indicating a small decrease in CXCR2 trafficking from the trans-Golgi network to the plasma membrane. AngII has no effect and both CXCL8 and AngII combined has the same effect as CXCL8 only.

Referring now to FIG. 4N, in the presence of AT1R, CXCL8 treatment again results in a small decrease in the proximity of CXCR2/Rluc8 to Venus/Rab8, although it is perhaps less pronounced than observed in the absence of AT1R. Strikingly, AngII only treatment results in a clear increase in the proximity of CXCR2/Rluc8 to Venus/Rab8. Treatment with both AngII and CXCL8 still results in a clear increase in proximity, slightly reduced compared to AngII only. These data indicate that activation of AT1R by AngII promotes forward trafficking of CXCR2 from the trans-Golgi network to the plasma membrane, consistent with the observed increase in CXCR2/Rluc8 proximity to Venus/Kras upon activation of AT1R by AngII.

Referring now to FIG. 4O, in the absence of AT1R, CXCL8 treatment results in an increase in the proximity of CXCR2/Rluc8 to Venus/Rab9, indicating an increase in CXCR2 trafficking from the late endosome to the trans-Golgi network. AngII has no effect and both CXCL8 and AngII combined has the same effect as CXCL8 only.

Referring now to FIG. 4P, in the presence of AT1R, CXCL8 treatment again results in an increase in the proximity of CXCR2/Rluc8 to Venus/Rab9, indicating an increase in CXCR2 trafficking from the late endosome to the trans-Golgi network. AngII only results in a small increase, however, co-treatment with AngII slightly reduces the effect of CXCL8.

Referring now to FIG. 4Q, in the absence of AT1R, CXCL8 treatment results in an increase in the proximity of CXCR2/Rluc8 to Venus/Rab11a, indicating an increase in CXCR2 trafficking through recycling endosomes. AngII has no effect and both CXCL8 and AngII combined has the same effect as CXCL8 only.

Referring now to FIG. 4R, in the presence of AT1R, CXCL8 treatment again results in an increase in the proximity of CXCR2/Rluc8 to Venus/Rab11a, again indicating an increase in CXCR2 trafficking through recycling endosomes. There is barely any effect of AngII only, however, co-treatment with AngII reduces the effect of CXCL8.

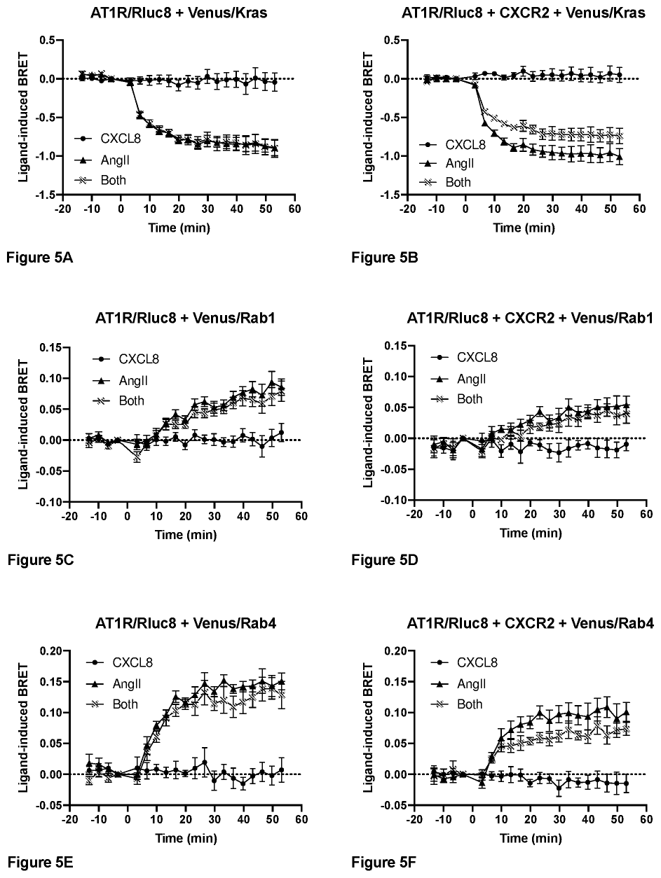

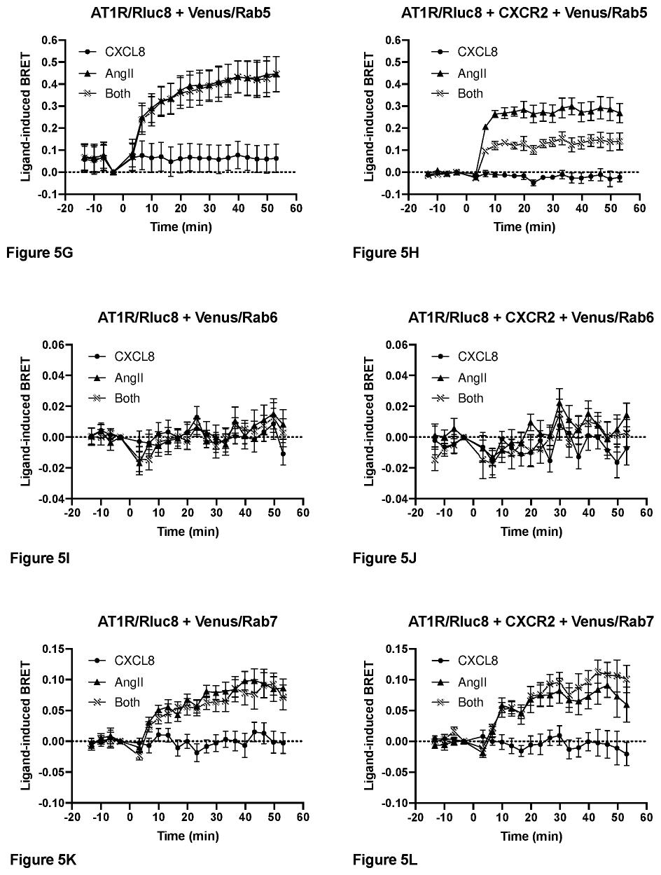

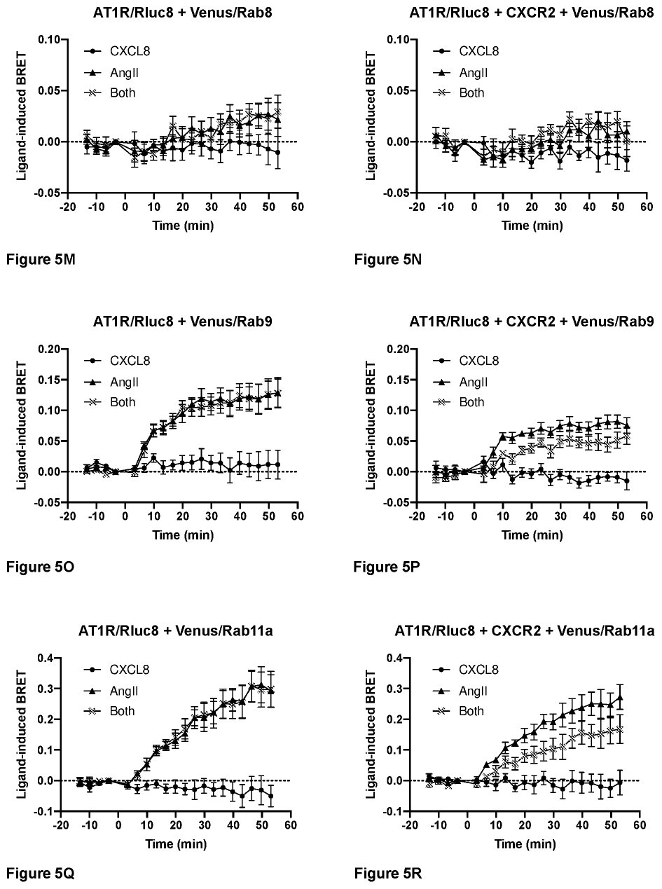

Referring now to FIG. 5A, in the absence of CXCR2, AngII treatment results in a marked reduction in the proximity of AT1R/Rluc8 to plasma membrane marker Venus/Kras, indicating that AT1R is internalized into the cell. CXCL8 has no effect and both CXCL8 and AngII combined has the same effect as AngII only.

Referring now to FIG. 5B, in the presence of CXCR2, AngII treatment again results in a marked reduction in the proximity of AT1R/Rluc8 to plasma membrane marker Venus/Kras, again indicating that AT1R is internalized into the cell. In contrast to that observed in the other orientation (FIG. 4B), AngII treatment does not result in a notable increase in proximity of AT1R/Rluc8 and Venus/Kras, indicating that activation of the CXCR2 by CXCL8 does not increase the amount of AT1R on the plasma membrane. Therefore, the effect clearly observed in FIG. 4B does not appear to be reciprocal. Treatment with both CXCL8 and AngII resulted in less AT1R internalization compared with AngII only.

Referring now to FIG. 5C, in the absence of CXCR2, AngII treatment results in an increase in the proximity of AT1R/Rluc8 to Venus/Rab1, indicating an increase in AT1R trafficking from the endoplasmic reticulum to the cis-Golgi. CXCL8 has no effect and both CXCL8 and AngII combined has the same effect as AngII only.

Referring now to FIG. 5D, in the presence of CXCR2, AngII treatment again results in an increase in the proximity of AT1R/Rluc8 to Venus/Rab1, again indicating an increase in AT1R trafficking from the endoplasmic reticulum to the cis-Golgi. There is barely any effect of CXCL8 only (possibly a slight decrease), and co-treatment with CXCL8 has minimal impact on the effect of AngII.

Referring now to FIG. 5E, in the absence of CXCR2, AngII treatment results in an increase in the proximity of AT1R/Rluc8 to Venus/Rab4, indicating an increase in early endosome recycling of AT1R. CXCL8 has no effect and both CXCL8 and AngII combined has the same effect as AngII only.

Referring now to FIG. 5F, in the presence of CXCR2, AngII treatment again results in an increase in the proximity of AT1R/Rluc8 to Venus/Rab4, again indicating an increase in early endosome recycling of AT1R. There is barely any effect of CXCL8 only (possibly a slight decrease), although co-treatment with CXCL8 reduces the effect of AngII.

Referring now to FIG. 5G, in the absence of CXCR2, AngII treatment results in a substantial increase in the proximity of AT1R/Rluc8 to Venus/Rab5, indicating a substantial increase in AT1R in early endosomes. CXCL8 has no effect and both CXCL8 and AngII combined has the same effect as AngII only.

Referring now to FIG. 5H, in the presence of CXCR2, AngII treatment again results in a substantial increase in the proximity of AT1R/Rluc8 to Venus/Rab5, indicating a substantial increase in AT1R in early endosomes. CXCL8 only has no discernible effect, however, co-treatment with CXCL8 appears to substantially reduce the effect of AngII. This is consistent with reduced AngII-induced internalization of AT1R in the presence of CXCR2 activated by CXCL8, resulting in increased CXCR2 on the plasma membrane as observed with the Kras data.

Referring now to FIG. 5I, in the absence of CXCR2, AngII treatment has little effect on the proximity of AT1R/Rluc8 to Venus/Rab6 (possibly a very small and transient decrease), indicating little effect on AT1R trafficking through the Golgi apparatus to the trans-Golgi network. CXCL8 has no effect and both CXCL8 and AngII combined has the same effect as AngII only.

Referring now to FIG. 5J, the presence of CXCR2 makes very little difference compared to the results observed in the absence of CXCR2 with respect to AT1R/Rluc8 proximity to Venus/Rab6.

Referring now to FIG. 5K, in the absence of CXCR2, AngII treatment results in an increase in the proximity of AT1R/Rluc8 to Venus/Rab7, indicating an increase in AT1R in late endosomes/lysosomes. CXCL8 has no effect and both CXCL8 and AngII combined has the same effect as AngII only.

Referring now to FIG. 5L, in the presence of CXCR2, AngII treatment again results in an increase in the proximity of AT1R/Rluc8 to Venus/Rab7, again indicating an increase in AT1R in late endosomes/lysosomes. There is no effect of CXCL8 only, and co-treatment with CXCL8 possibly increases the effect observed with AngII at later time-points).

Referring now to FIG. 5M, in the absence of CXCR2, AngII treatment results in a small increase at later time-points in the proximity of AT1R/Rluc8 to Venus/Rab8, indicating a small effect on AT1R trafficking from the trans-Golgi network to the plasma membrane. CXCL8 has no effect and both CXCL8 and AngII combined has the same effect as AngII only.

Referring now to FIG. 5N, the presence of CXCR2 makes little difference compared to the results observed in the absence of CXCR2 with respect to AT1R/Rluc8 proximity to Venus/Rab8. If anything, CXCL8 reduces the proximity, AngII has less of an effect, and both CXCL8 and AngII combined again has the same effect as AngII only.

Referring now to FIG. 5O, in the absence of CXCR2, AngII treatment results in an increase in the proximity of AT1R/Rluc8 to Venus/Rab9, indicating an increase in AT1R trafficking from the late endosome to the trans-Golgi network. CXCL8 has no effect and both CXCL8 and AngII combined has the same effect as AngII only.

Referring now to FIG. 5P, in the presence of CXCR2, AngII treatment again results in an increase in the proximity of AT1R/Rluc8 to Venus/Rab9, again indicating an increase in AT1R trafficking from the late endosome to the trans-Golgi network. There is barely any effect of CXCL8 only, although co-treatment with CXCL8 reduces the effect of AngII.

Referring now to FIG. 5Q, in the absence of CXCR2, AngII treatment results in a substantial increase in the proximity of AT1R/Rluc8 to Venus/Rab11a, indicating a substantial increase in AT1R trafficking through recycling endosomes. CXCL8 has no effect and both CXCL8 and AngII combined has the same effect as AngII only.

Referring now to FIG. 5R, in the presence of CXCR2, AngII treatment again results in a substantial increase in the proximity of AT1R/Rluc8 to Venus/Rab11a, indicating a substantial increase in AT1R trafficking through recycling endosomes. CXCL8 only has no discernible effect, however, co-treatment with CXCL8 reduces the effect of AngII.

This example demonstrates that activation of AT1R increases the expression of CXCR2 on the plasma membrane, by reducing the amount of CXCR2 internalization and/or by increasing the forward trafficking of CXCR2 from the trans-Golgi network up to the plasma membrane. Furthermore, this increase in CXCR2 expression at the plasma membrane as a consequence of AT1R activation is consistent with the data showing a more than additive effect of treatment with both CXCL8 and AngII on Gi coupling (as demonstrated by proximity to the biosensor mGsi) when both AT1R and CXCR2 are present. Moreover, this increase in CXCR2 expression at the plasma membrane as a consequence of AT1R activation is consistent with the potentiation of the phospholipase C activity (as demonstrated by potentiation of IP-1 accumulation) observed when cells are co-treated with a fixed concentration of CXCL8 in addition to a range of concentrations of AngII.

This example also demonstrates that activation of CXCR2 reduces the amount of AT1R internalization, in this regard reciprocating the effect observed on CXCR2 when AT1R is activated. In contrast however, CXCR2 activation does not increase the forward trafficking of AT1R from the trans-Golgi network up to the plasma membrane, thereby in this regard not reciprocating the effect observed on CXCR2 when AT1R is activated.

### Example 4

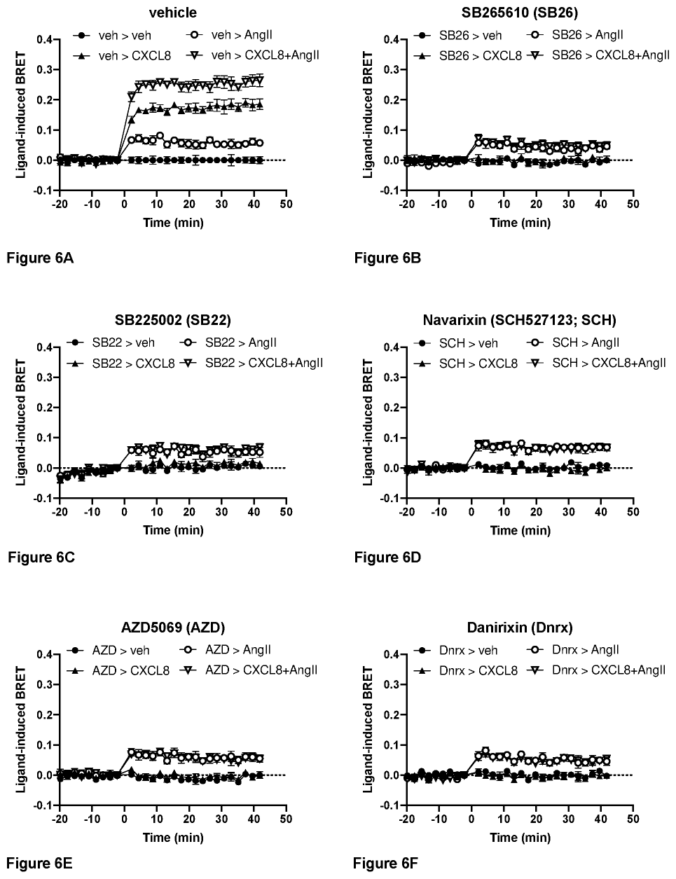

Referring now to FIGS. 6-15, BRET signals were measured from cells transiently expressing CXCR2/Rluc8, Venus/mGsi and HA-AT1R following pre-treatment with vehicle or antagonists/inverse agonists (10 μM) followed 50 minutes later by treatment with either 10 nM CXCL8 or 100 nM AngII only or both CXCL8 and AngII combined (10-fold lower concentrations than used to generate the data for FIG. 1B). Prior to agonist treatment (added at 0 minutes), a baseline BRET signal was recorded for each of the combinations for 20 minutes. Data were calculated as ligand-induced BRET relative to the ‘vehicle followed by vehicle’ (veh>veh) data set.

Referring now to FIG. 6A, cells were pre-treated with vehicle as a control to establish the ‘vehicle followed by vehicle’ baseline and show the effect of CXCL8 only, AngII only and both CXCL8 and AngII combined at concentrations used for the following experiments with antagonists/inverse agonists.

Referring now to FIGS. 6B, 6C, 6D, 6E and 6F, cells were pre-treated with CXCR2 allosteric inverse agonist SB265610 (FIG. 6B), CXCR2 antagonist SB225002 (FIG. 6C), CXCR2 inverse agonist navarixin (SCH527123; FIG. 6D), CXCR2 antagonist AZD5069 (FIG. 6E) or CXCR2 antagonist danirixin (FIG. 6F), resulting in the effect of CXCL8 being completely inhibited but the effect of AngII not being altered. The effect of CXCL8 and AngII combined is indistinguishable from that observed with AngII only in each case.

Referring now to FIG. 7A, cells were pre-treated with AT1R antagonist irbesartan, resulting in the effect of AngII being completely inhibited but the effect of CXCL8 not being altered. The effect of CXCL8 and AngII combined is indistinguishable from that observed with CXCL8 only.

Referring now to FIGS. 7B, 7C, 7D, 7E and 7F, cells were pre-treated with both AT1R antagonist irbesartan and either CXCR2 allosteric inverse agonist SB265610 (FIG. 7B), CXCR2 antagonist SB225002 (FIG. 7C), CXCR2 inverse agonist navarixin (SCH527123; FIG. 7D), CXCR2 antagonist AZD5069 (FIG. 7E) or CXCR2 antagonist danirixin (FIG. 7F), resulting in the effect of CXCL8 only, the effect of AngII only or the effect of both combined being completely inhibited.

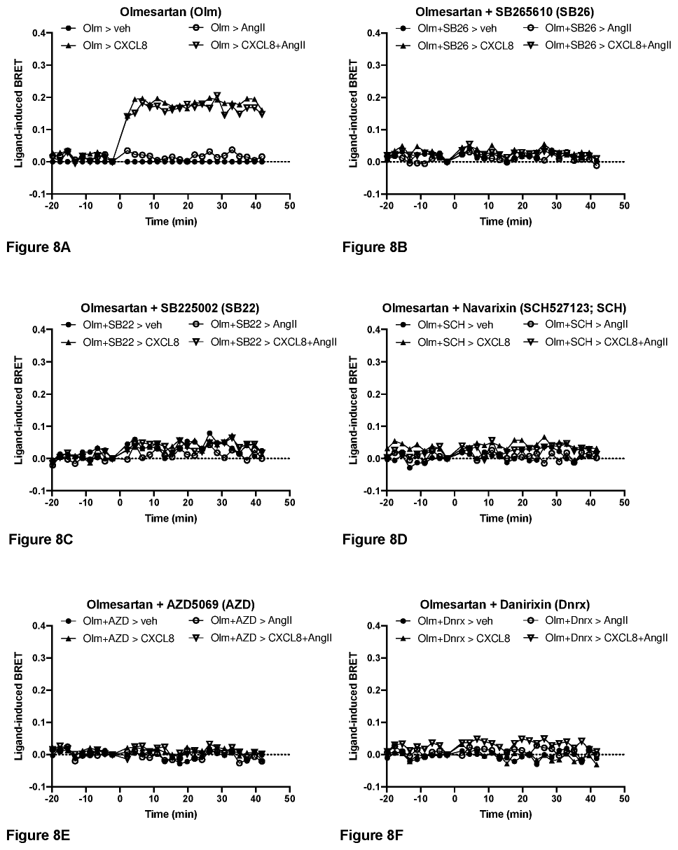

Referring now to FIG. 8A, cells were pre-treated with AT1R antagonist olmesartan, resulting in the effect of AngII being completely inhibited but the effect of CXCL8 not being altered. The effect of CXCL8 and AngII combined is indistinguishable from that observed with CXCL8 only.

Referring now to FIGS. 8B, 8C, 8D, 8E and 8F, cells were pre-treated with both AT1R antagonist olmesartan and either CXCR2 allosteric inverse agonist SB265610 (FIG. 8B), CXCR2 antagonist SB225002 (FIG. 8C), CXCR2 inverse agonist navarixin (SCH527123; FIG. 8D), CXCR2 antagonist AZD5069 (FIG. 8E) or CXCR2 antagonist danirixin (FIG. 8F), resulting in the effect of CXCL8 only, the effect of AngII only or the effect of both combined being completely inhibited.

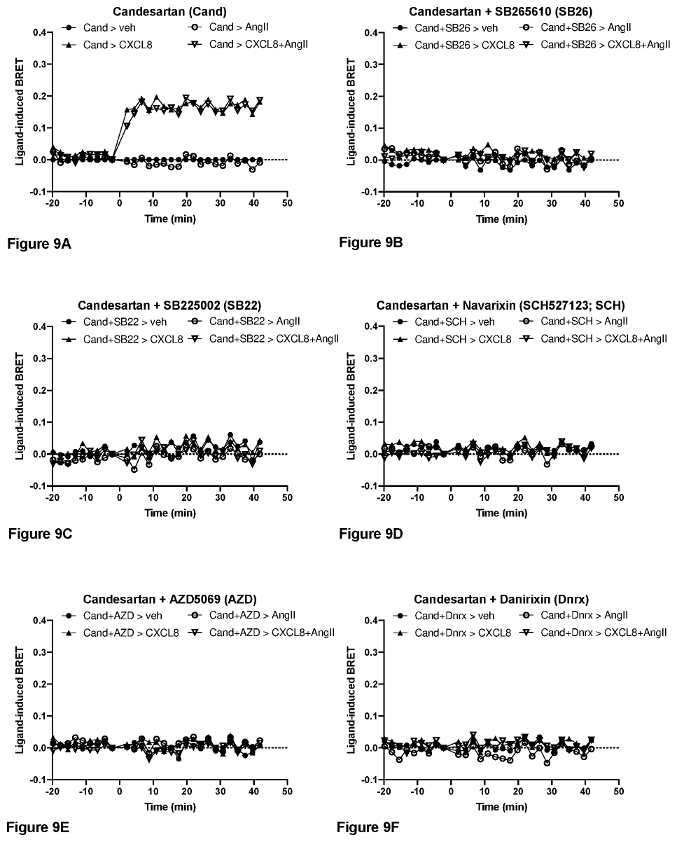

Referring now to FIG. 9A, cells were pre-treated with AT1R antagonist candesartan, resulting in the effect of AngII being completely inhibited but the effect of CXCL8 not being altered. The effect of CXCL8 and AngII combined is indistinguishable from that observed with CXCL8 only.

Referring now to FIGS. 9B, 9C, 9D, 9E and 9F, cells were pre-treated with both AT1R antagonist candesartan and either CXCR2 allosteric inverse agonist SB265610 (FIG. 9B), CXCR2 antagonist SB225002 (FIG. 9C), CXCR2 inverse agonist navarixin (SCH527123; FIG. 9D), CXCR2 antagonist AZD5069 (FIG. 9E) or CXCR2 antagonist danirixin (FIG. 9F), resulting in the effect of CXCL8 only, the effect of AngII only or the effect of both combined being completely inhibited.

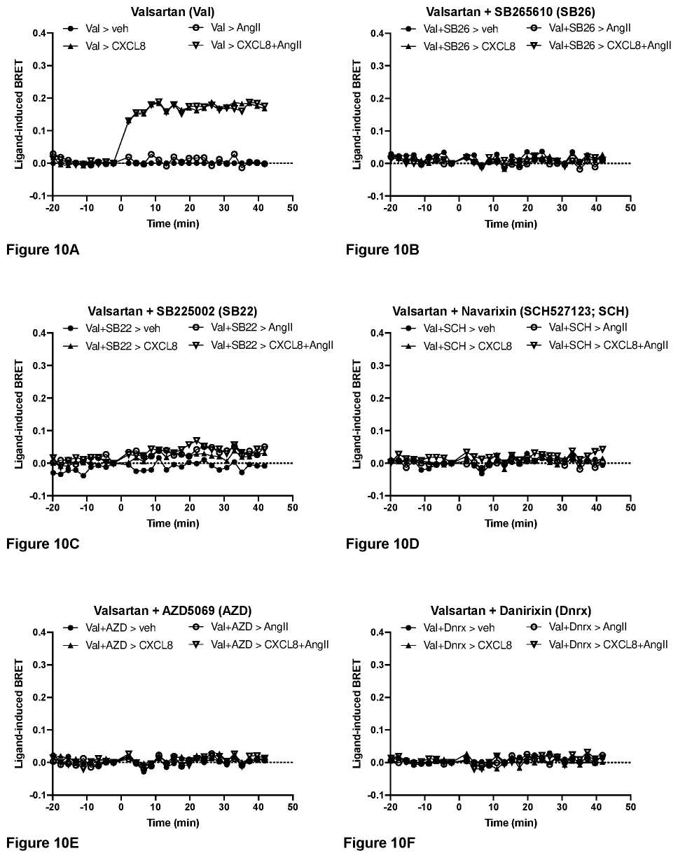

Referring now to FIG. 10A, cells were pre-treated with AT1R antagonist valsartan, resulting in the effect of AngII being completely inhibited but the effect of CXCL8 not being altered. The effect of CXCL8 and AngII combined is indistinguishable from that observed with CXCL8 only.

Referring now to FIGS. 10B, 100, 10D, 10E and 10F, cells were pre-treated with both AT1R antagonist valsartan and either CXCR2 allosteric inverse agonist SB265610 (FIG. 10B), CXCR2 antagonist SB225002 (FIG. 100), CXCR2 inverse agonist navarixin (SCH527123; FIG. 10D), CXCR2 antagonist AZD5069 (FIG. 10E) or CXCR2 antagonist danirixin (FIG. 10F), resulting in the effect of CXCL8 only, the effect of AngII only or the effect of both combined being completely inhibited.

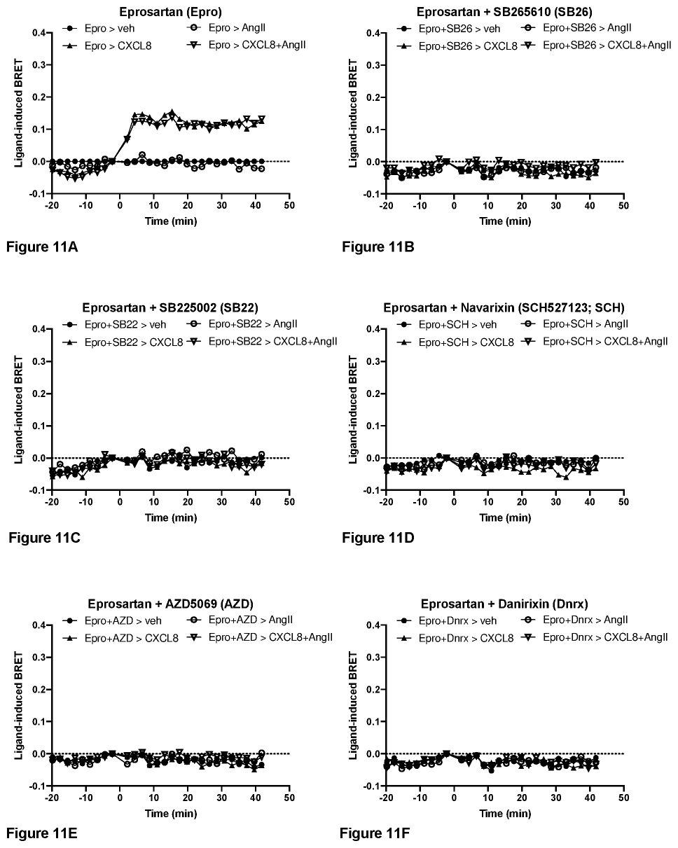

Referring now to FIG. 11A, cells were pre-treated with AT1R antagonist eprosartan, resulting in the effect of AngII being completely inhibited but the effect of CXCL8 not being altered. The effect of CXCL8 and AngII combined is indistinguishable from that observed with CXCL8 only.

Referring now to FIGS. 11B, 11C, 11D, 11E and 11F, cells were pre-treated with both AT1R antagonist eprosartan and either CXCR2 allosteric inverse agonist SB265610 (FIG. 11B), CXCR2 antagonist SB225002 (FIG. 11C), CXCR2 inverse agonist navarixin (SCH527123; FIG. 11D), CXCR2 antagonist AZD5069 (FIG. 11E) or CXCR2 antagonist danirixin (FIG. 11F), resulting in the effect of CXCL8 only, the effect of AngII only or the effect of both combined being completely inhibited.

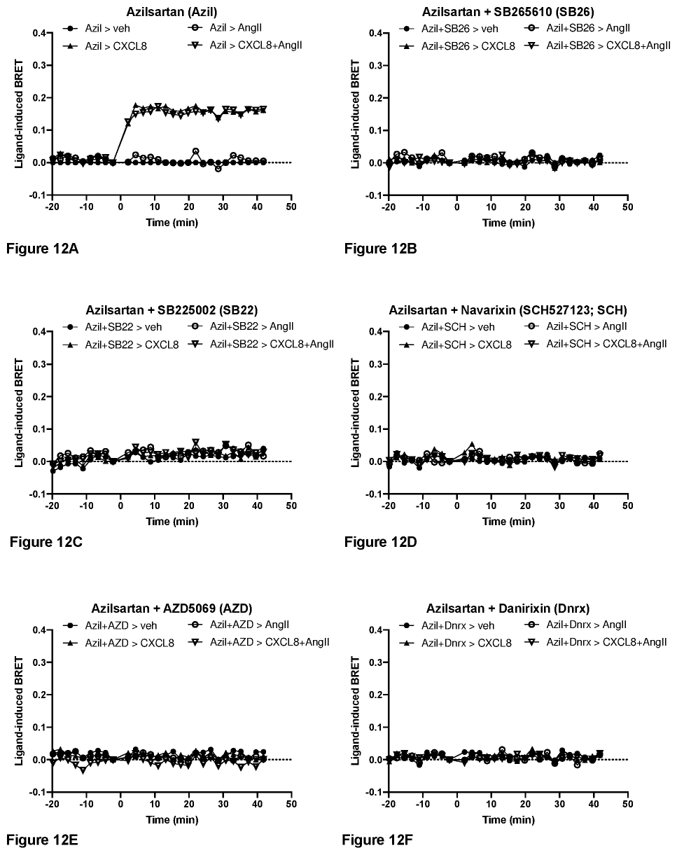

Referring now to FIG. 12A, cells were pre-treated with AT1R antagonist azilsartan, resulting in the effect of AngII being completely inhibited but the effect of CXCL8 not being altered. The effect of CXCL8 and AngII combined is indistinguishable from that observed with CXCL8 only.

Referring now to FIGS. 12B, 12C, 12D, 12E and 12F, cells were pre-treated with both AT1R antagonist azilsartan and either CXCR2 allosteric inverse agonist SB265610 (FIG. 12B), CXCR2 antagonist SB225002 (FIG. 12C), CXCR2 inverse agonist navarixin (SCH527123; FIG. 12D), CXCR2 antagonist AZD5069 (FIG. 12E) or CXCR2 antagonist danirixin (FIG. 12F), resulting in the effect of CXCL8 only, the effect of AngII only or the effect of both combined being completely inhibited.

Referring now to FIG. 13A, cells were pre-treated with AT1R antagonist losartan, resulting in the effect of AngII being completely inhibited but the effect of CXCL8 not being altered. The effect of CXCL8 and AngII combined is indistinguishable from that observed with CXCL8 only.

Referring now to FIGS. 13B, 13C, 13D, 13E and 13F, cells were pre-treated with both AT1R antagonist losartan and either CXCR2 allosteric inverse agonist SB265610 (FIG. 13B), CXCR2 antagonist SB225002 (FIG. 13C), CXCR2 inverse agonist navarixin (SCH527123; FIG. 13D), CXCR2 antagonist AZD5069 (FIG. 13E) or CXCR2 antagonist danirixin (FIG. 13F), resulting in the effect of CXCL8 only, the effect of AngII only or the effect of both combined being completely inhibited.

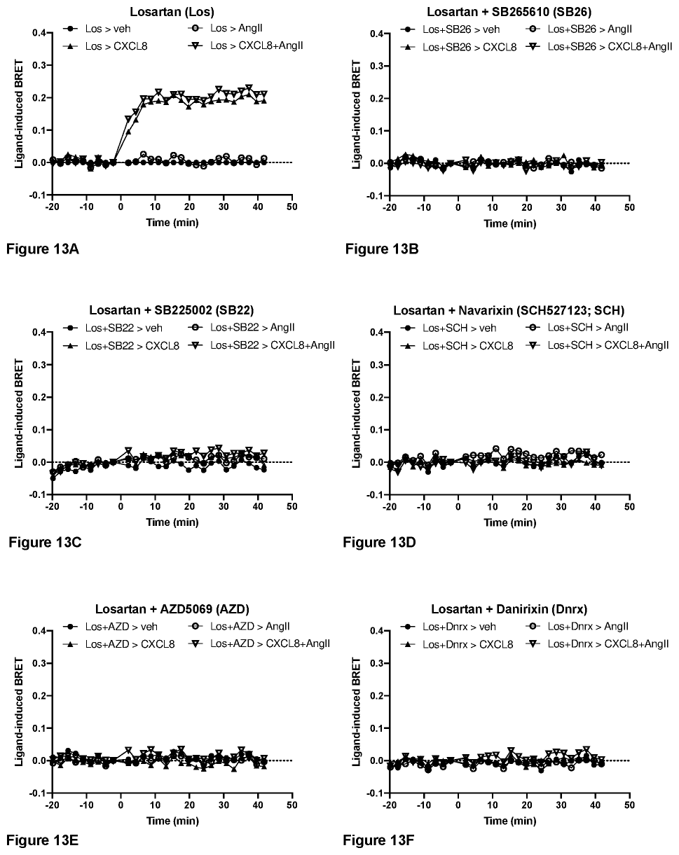

Referring now to FIG. 14A, cells were pre-treated with AT1R antagonist EXP3174, the active metabolite of losartan, resulting in the effect of AngII being completely inhibited but the effect of CXCL8 not being altered. The effect of CXCL8 and AngII combined is indistinguishable from that observed with CXCL8 only.

Referring now to FIGS. 14B, 14C, 14D, 14E and 14F, cells were pre-treated with both AT1R antagonist EXP3174, the active metabolite of losartan, and either CXCR2 allosteric inverse agonist SB265610 (FIG. 14B), CXCR2 antagonist SB225002 (FIG. 14C), CXCR2 inverse agonist navarixin (SCH527123; FIG. 14D), CXCR2 antagonist AZD5069 (FIG. 14E) or CXCR2 antagonist danirixin (FIG. 14F), resulting in the effect of CXCL8 only, the effect of AngII only or the effect of both combined being completely inhibited.

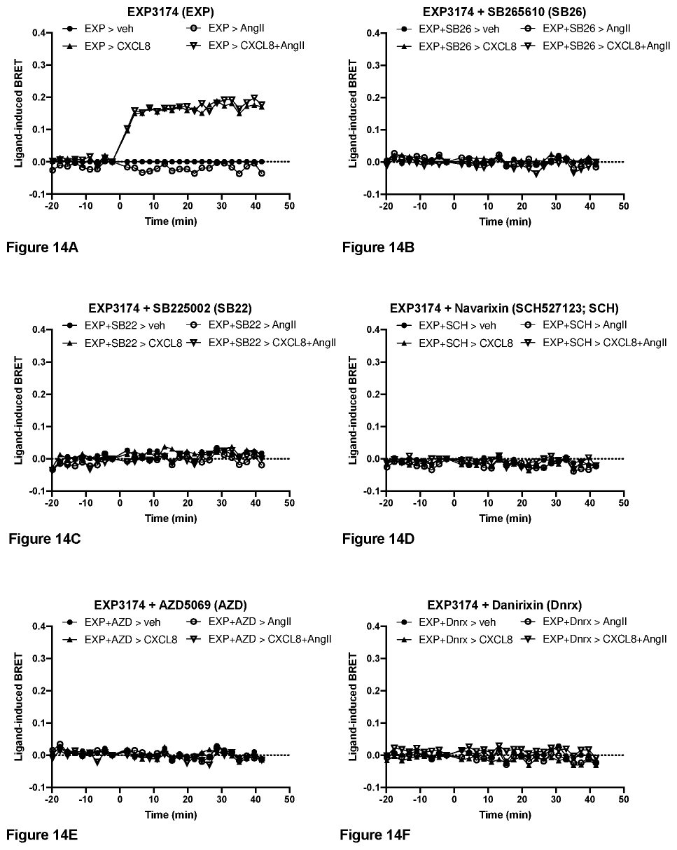

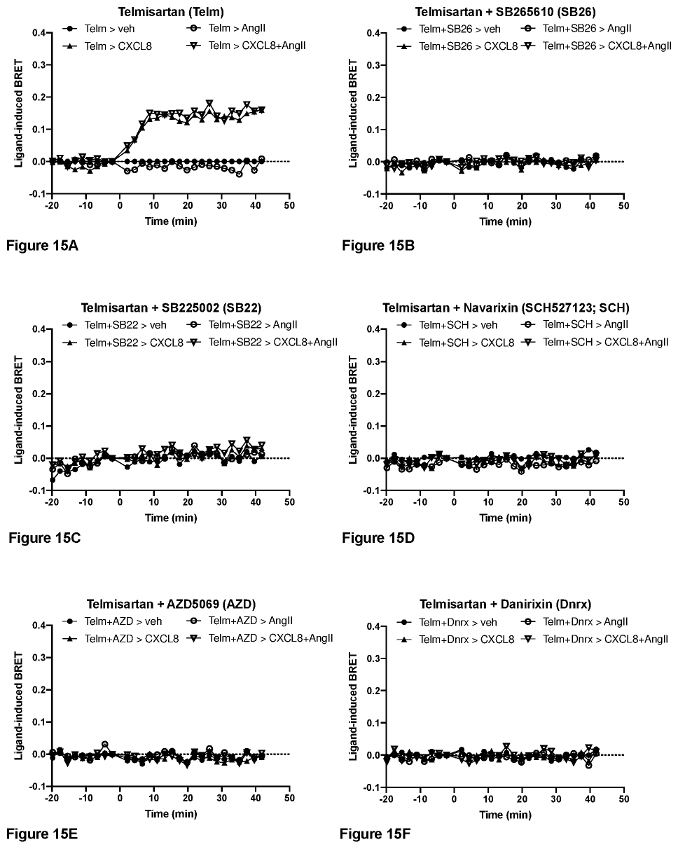

Referring now to FIG. 15A, cells were pre-treated with AT1R antagonist telmisartan, resulting in the effect of AngII being completely inhibited but the effect of CXCL8 not being altered. The effect of CXCL8 and AngII combined is indistinguishable from that observed with CXCL8 only.

Referring now to FIGS. 15B, 15C, 15D, 15E and 15F, cells were pre-treated with both AT1R antagonist telmisartan and either CXCR2 allosteric inverse agonist SB265610 (FIG. 15B), CXCR2 antagonist SB225002 (FIG. 15C), CXCR2 inverse agonist navarixin (SCH527123; FIG. 15D), CXCR2 antagonist AZD5069 (FIG. 15E) or CXCR2 antagonist danirixin (FIG. 15F), resulting in the effect of CXCL8 only, the effect of AngII only or the effect of both combined being completely inhibited.

This example demonstrates that Gi coupling (as indicated by an increase in the CXCR2/Rluc8 proximity to the Venus/mGsi biosensor) upon activation of both CXCR2 and AT1R in the AT1R-CXCR2 heteromer is completely inhibited by addition of an AT1R antagonist in combination with a CXCR2 antagonist, CXCR2 inverse agonist or CXCR2 allosteric inverse agonist (as an example of a CXCR2 negative allosteric modulator that is also a CXCR2 inverse agonist).

### Example 5

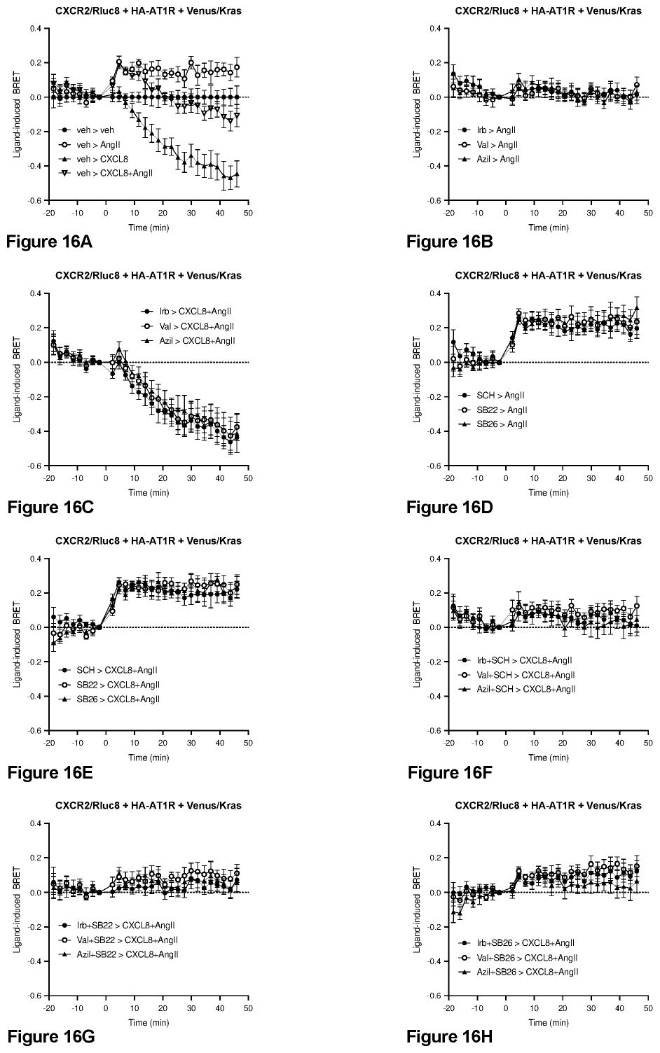

Referring now to FIG. 16, BRET signals were measured from cells transiently expressing CXCR2/Rluc8, Venus/Kras and HA-AT1R following pre-treatment with vehicle or antagonists/inverse agonists (10 μM) followed 50 minutes later by treatment with either 10 nM CXCL8 or 100 nM AngII only or both CXCL8 and AngII combined (10-fold lower concentrations than used to generate the data for FIG. 4B) as indicated. Prior to agonist treatment (added at 0 minutes), a baseline BRET signal was recorded for each of the combinations for 20 minutes. Data were calculated as ligand-induced BRET relative to the ‘vehicle followed by vehicle’ (veh>veh) data set.

Referring now to FIG. 16A, as observed in FIG. 4B, CXCL8 treatment results in a marked reduction in the proximity of CXCR2/Rluc8 to plasma membrane marker Venus/Kras, indicating that CXCR2 is internalized into the cell. However, in contrast, AngII treatment results in increased proximity of CXCR2/Rluc8 and Venus/Kras, providing evidence for activation of the AT1R by AngII increasing the amount of CXCR2 on the plasma membrane. Treatment with both CXCL8 and AngII resulted in less CXCR2 net internalization compared with CXCL8 only, which may be the net effect of internalization induced by CXCL8 and forward trafficking of CXCR2 to the plasma membrane induced by AT1R activation by AngII. Cells were pre-treated with vehicle as a control to establish the ‘vehicle followed by vehicle’ baseline and show the effect of CXCL8 only, AngII only and both CXCL8 and AngII combined at concentrations used for the following experiments with antagonists/inverse agonists.

Referring now to FIG. 16B, pre-treatment with AT1R antagonist irbesartan, valsartan or azilsartan completely inhibited the effect of AngII.

Referring now to FIG. 16C, pre-treatment with AT1R antagonist irbesartan, valsartan or azilsartan resulted in the effect of both CXCL8 and AngII combined being indistinguishable from the effect observed with CXCL8 only when the cells are pre-treated with vehicle.

Referring now to FIG. 16D, pre-treatment with CXCR2 inverse agonist navarixin (SCH527123), CXCR2 antagonist SB225002 or CXCR2 allosteric inverse agonist SB265610 did not alter the effect of AngII.

Referring now to FIG. 16E, pre-treatment with CXCR2 inverse agonist navarixin (SCH527123), CXCR2 antagonist SB225002 or CXCR2 allosteric inverse agonist SB265610 resulted in the effect of both CXCL8 and AngII combined being indistinguishable from the effect observed with AngII only when the cells are pre-treated with vehicle.

Referring now to FIG. 16F, pre-treatment with CXCR2 inverse agonist navarixin (SCH527123) in combination with AT1R antagonist irbesartan, valsartan or azilsartan resulted in the effect of both CXCL8 and AngII combined being inhibited.

Referring now to FIG. 16G, pre-treatment with CXCR2 antagonist SB225002 in combination with AT1R antagonist irbesartan, valsartan or azilsartan resulted in the effect of both CXCL8 and AngII combined being inhibited.

Referring now to FIG. 16H, pre-treatment with CXCR2 allosteric inverse agonist SB265610 in combination with AT1R antagonist irbesartan, valsartan or azilsartan resulted in the effect of both CXCL8 and AngII combined being inhibited.

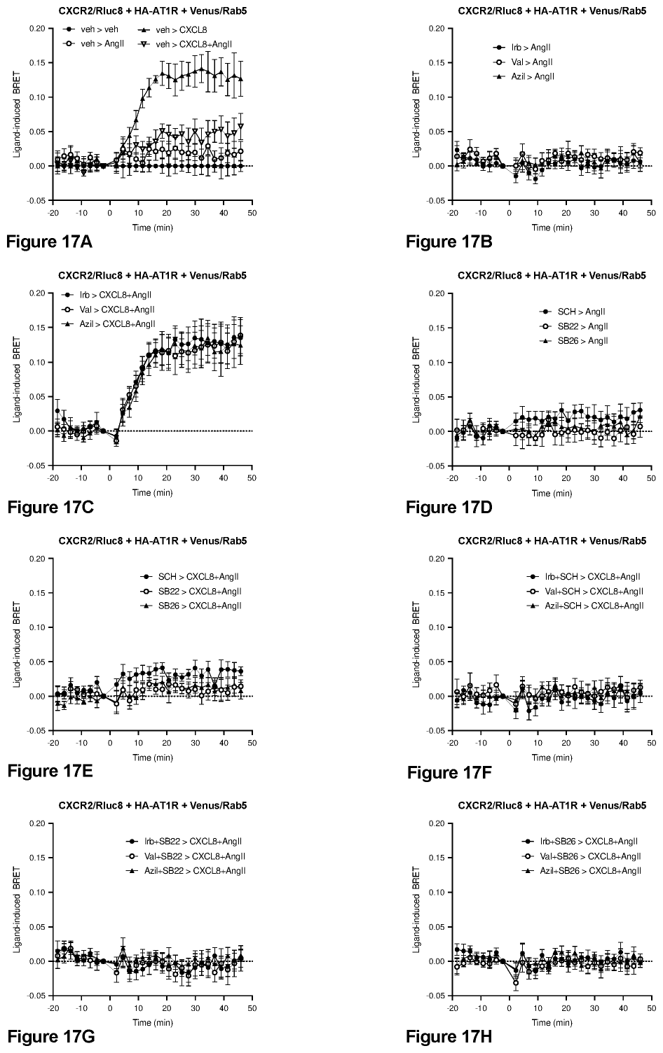

Referring now to FIG. 17A, as observed in FIG. 4H, CXCL8 treatment results in a substantial increase in the proximity of CXCR2/Rluc8 to Venus/Rab5, indicating a substantial increase in CXCR2 in early endosomes. AngII only results in a very small increase in CXCR2 in early endosomes, however, co-treatment with AngII substantially reduces the effect of CXCL8. This is consistent with reduced CXCL8-induced internalization of CXCR2 in the presence of AT1R activated by AngII, resulting in increased CXCR2 on the plasma membrane as observed with the Kras data. Cells were pre-treated with vehicle as a control to establish the ‘vehicle followed by vehicle’ baseline and show the effect of CXCL8 only, AngII only and both CXCL8 and AngII combined at concentrations used for the following experiments with antagonists/inverse agonists.

Referring now to FIG. 17B, pre-treatment with AT1R antagonist irbesartan, valsartan or azilsartan completely inhibited the effect of AngII.

Referring now to FIG. 17C, pre-treatment with AT1R antagonist irbesartan, valsartan or azilsartan resulted in the effect of both CXCL8 and AngII combined being indistinguishable from the effect observed with CXCL8 only when the cells are pre-treated with vehicle.

Referring now to FIG. 17D, pre-treatment with CXCR2 inverse agonist navarixin (SCH527123) did not alter the effect of AngII, whereas pre-treatment with CXCR2 antagonist SB225002 or CXCR2 allosteric inverse agonist SB265610 did appear to inhibit the effect of AngII.

Referring now to FIG. 17E, pre-treatment with CXCR2 inverse agonist navarixin (SCH527123) resulted in the effect of both CXCL8 and AngII combined being indistinguishable from the effect observed with AngII only when the cells are pre-treated with vehicle. In contrast, pre-treatment with CXCR2 antagonist SB225002 or CXCR2 allosteric inverse agonist SB265610 appears to inhibit the effect of AngII as well as that of CXCL8.

Referring now to FIG. 17F, pre-treatment with CXCR2 inverse agonist navarixin (SCH527123) in combination with AT1R antagonist irbesartan, valsartan or azilsartan resulted in the effect of both CXCL8 and AngII combined being completely inhibited.

Referring now to FIG. 17G, pre-treatment with CXCR2 antagonist SB225002 in combination with AT1R antagonist irbesartan, valsartan or azilsartan resulted in the effect of both CXCL8 and AngII combined being completely inhibited.

Referring now to FIG. 17H, pre-treatment with CXCR2 allosteric inverse agonist SB265610 in combination with AT1R antagonist irbesartan, valsartan or azilsartan resulted in the effect of both CXCL8 and AngII combined being completely inhibited.

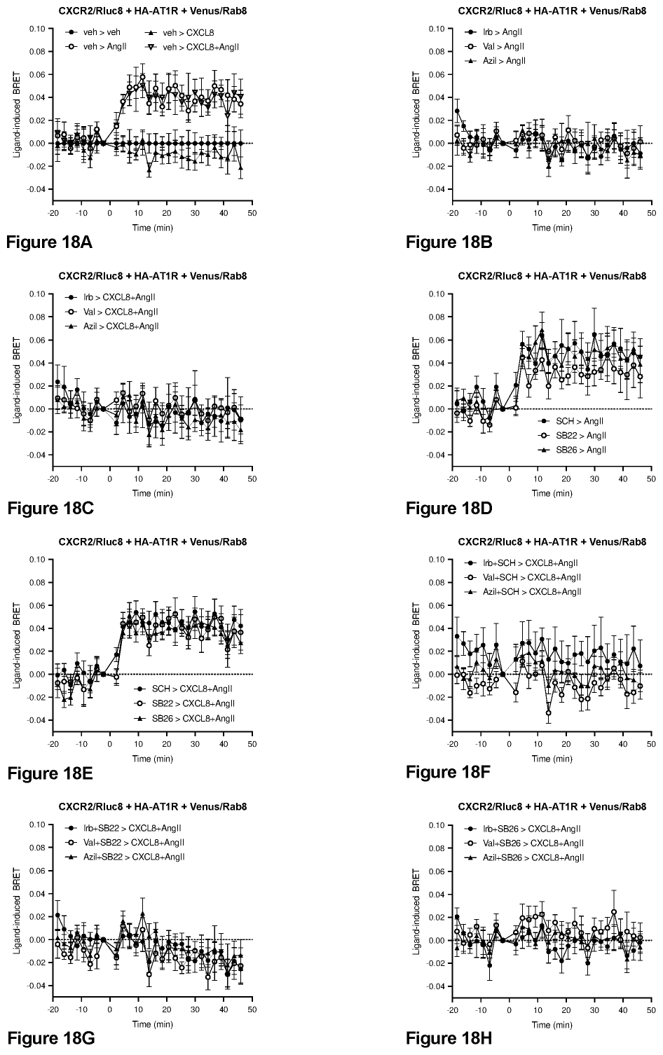

Referring now to FIG. 18A, as observed in FIG. 4N, CXCL8 treatment results in a small decrease in the proximity of CXCR2/Rluc8 to Venus/Rab8. Strikingly, AngII only treatment results in a clear increase in the proximity of CXCR2/Rluc8 to Venus/Rab8, as does treatment with both AngII and CXCL8 combined. These data indicate that activation of AT1R by AngII promotes forward trafficking of CXCR2 from the trans-Golgi network to the plasma membrane, consistent with the observed increase in CXCR2/Rluc8 proximity to Venus/Kras upon activation of AT1R by AngII. Cells were pre-treated with vehicle as a control to establish the ‘vehicle followed by vehicle’ baseline and show the effect of CXCL8 only, AngII only and both CXCL8 and AngII combined at concentrations used for the following experiments with antagonists/inverse agonists.

Referring now to FIG. 18B, pre-treatment with AT1R antagonist irbesartan, valsartan or azilsartan completely inhibited the effect of AngII.

Referring now to FIG. 18C, pre-treatment with AT1R antagonist irbesartan, valsartan or azilsartan resulted in the effect of both CXCL8 and AngII combined being completely inhibited.

Referring now to FIG. 18D, pre-treatment with CXCR2 inverse agonist navarixin (SCH527123), CXCR2 antagonist SB225002 or CXCR2 allosteric inverse agonist SB265610 did not alter the effect of AngII.

Referring now to FIG. 18E, pre-treatment with CXCR2 inverse agonist navarixin (SCH527123), CXCR2 antagonist SB225002 or CXCR2 allosteric inverse agonist SB265610 resulted in the effect of both CXCL8 and AngII combined being indistinguishable from the effect observed with AngII only when the cells are pre-treated with vehicle.

Referring now to FIG. 18F, pre-treatment with CXCR2 inverse agonist navarixin (SCH527123) in combination with AT1R antagonist irbesartan, valsartan or azilsartan resulted in the effect of both CXCL8 and AngII combined being completely inhibited.

Referring now to FIG. 18G, pre-treatment with CXCR2 antagonist SB225002 in combination with AT1R antagonist irbesartan, valsartan or azilsartan resulted in the effect of both CXCL8 and AngII combined being completely inhibited.

Referring now to FIG. 18H, pre-treatment with CXCR2 allosteric inverse agonist SB265610 in combination with AT1R antagonist irbesartan, valsartan or azilsartan resulted in the effect of both CXCL8 and AngII combined being completely inhibited.

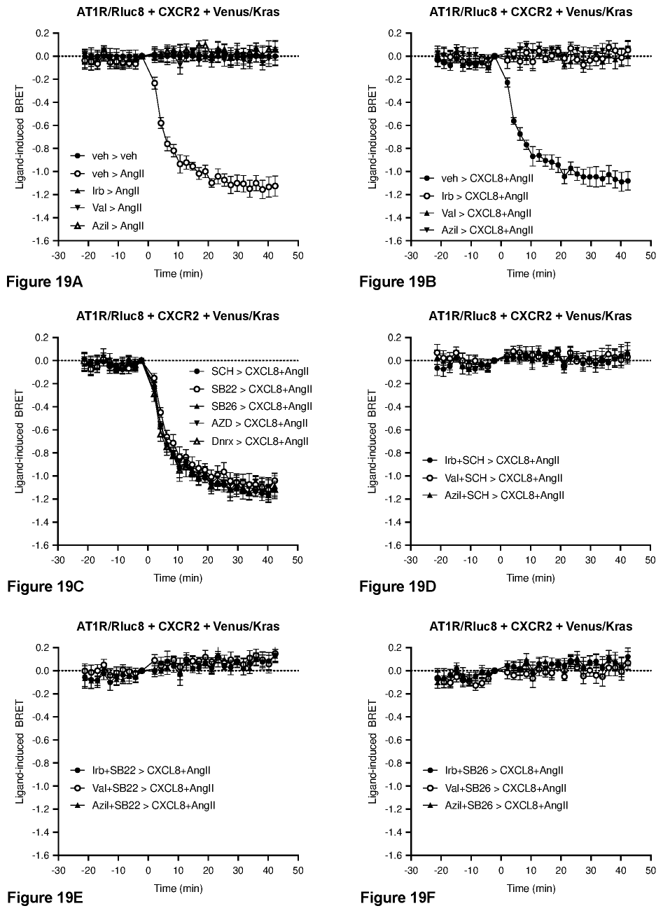

Referring now to FIG. 19A, AngII treatment results in a marked reduction in the proximity of AT1R/Rluc8 to plasma membrane marker Venus/Kras, indicating that AT1R is internalized into the cell. Some cells were pre-treated with vehicle as a control to establish the ‘vehicle followed by vehicle’ baseline and show the effect of AngII only at a concentration used for the following experiments with antagonists/inverse agonists. Pre-treatment with AT1R antagonist irbesartan, valsartan or azilsartan completely inhibited the effect of AngII.

Referring now to FIG. 19B, the effect of both CXCL8 and AngII combined was slightly less than the effect observed with AngII only (FIG. 19A). Pre-treatment with AT1R antagonist irbesartan, valsartan or azilsartan resulted in this effect being completely inhibited.

Referring now to FIG. 19C, pre-treatment with CXCR2 inverse agonist navarixin (SCH527123), CXCR2 antagonist SB225002, CXCR2 allosteric inverse agonist SB265610, CXCR2 antagonist AZD5069 or CXCR2 antagonist danirxin resulted in the effect observed following treatment with both CXCL8 and AngII combined being indistinguishable from the effect observed with AngII only when pre-treated with vehicle (FIG. 19A).

Referring now to FIG. 19D, pre-treatment with CXCR2 inverse agonist navarixin (SCH527123) in combination with AT1R antagonist irbesartan, valsartan or azilsartan resulted in the effect of both CXCL8 and AngII combined being completely inhibited.

Referring now to FIG. 19E, pre-treatment with CXCR2 antagonist SB225002 in combination with AT1R antagonist irbesartan, valsartan or azilsartan resulted in the effect of both CXCL8 and AngII combined being completely inhibited.

Referring now to FIG. 19F, pre-treatment with CXCR2 allosteric inverse agonist SB265610 in combination with AT1R antagonist irbesartan, valsartan or azilsartan resulted in the effect of both CXCL8 and AngII combined being completely inhibited.

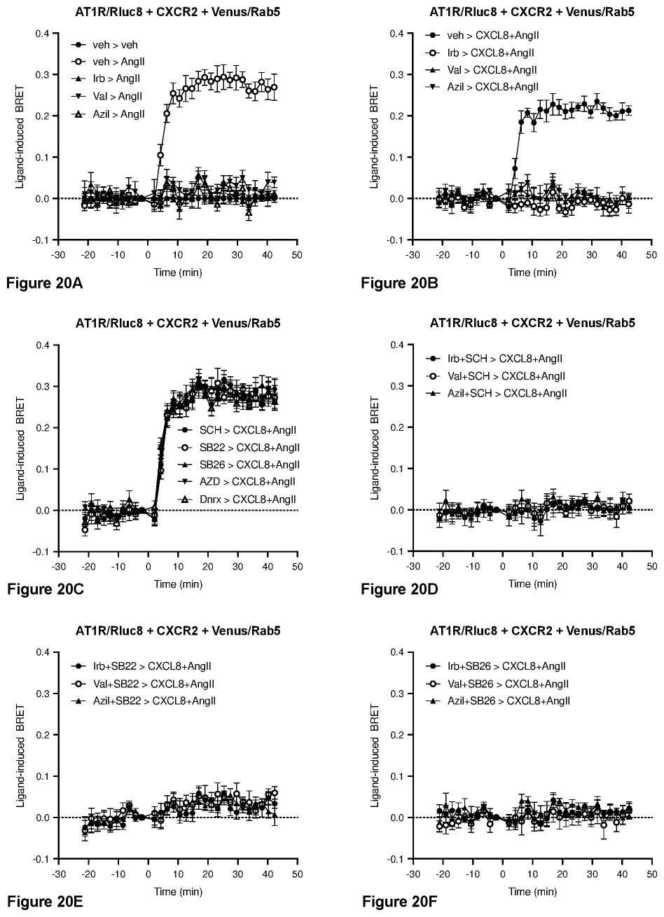

Referring now to FIG. 20A, AngII treatment results in a marked increase in the proximity of AT1R/Rluc8 to early endosome marker Venus/Rab5, indicating that AT1R is internalized into the cell. Some cells were pre-treated with vehicle as a control to establish the ‘vehicle followed by vehicle’ baseline and show the effect of AngII only at a concentration used for the following experiments with antagonists/inverse agonists. Pre-treatment with AT1R antagonist irbesartan, valsartan or azilsartan completely inhibited the effect of AngII.

Referring now to FIG. 20B, the effect of both CXCL8 and AngII combined was smaller than the effect observed with AngII only (FIG. 20A). Pre-treatment with AT1R antagonist irbesartan, valsartan or azilsartan resulted in this effect being completely inhibited.

Referring now to FIG. 20C, pre-treatment with CXCR2 inverse agonist navarixin (SCH527123), CXCR2 antagonist SB225002, CXCR2 allosteric inverse agonist SB265610, CXCR2 antagonist AZD5069 or CXCR2 antagonist danirxin resulted in the effect observed following treatment with both CXCL8 and AngII combined being indistinguishable from the effect observed with AngII only when pre-treated with vehicle (FIG. 19A).

Referring now to FIG. 20D, pre-treatment with CXCR2 inverse agonist navarixin (SCH527123) in combination with AT1R antagonist irbesartan, valsartan or azilsartan resulted in the effect of both CXCL8 and AngII combined being completely inhibited.

Referring now to FIG. 20E, pre-treatment with CXCR2 antagonist SB225002 in combination with AT1R antagonist irbesartan, valsartan or azilsartan resulted in the effect of both CXCL8 and AngII combined being almost completely inhibited.

Referring now to FIG. 20F, pre-treatment with CXCR2 allosteric inverse agonist SB265610 in combination with AT1R antagonist irbesartan, valsartan or azilsartan resulted in the effect of both CXCL8 and AngII combined being completely inhibited.

This example demonstrates that the effects on internalization observed upon activation of both CXCR2 and AT1R in the AT1R-CXCR2 heteromer are inhibited by addition of an AT1R antagonist in combination with a CXCR2 antagonist, CXCR2 inverse agonist or CXCR2 allosteric inverse agonist (as an example of a CXCR2 negative allosteric modulator that is also a CXCR2 inverse agonist).

Furthermore, this example demonstrates that the effects on CXCR2 forward trafficking are completely inhibited by addition of AT1R antagonists, further supporting the functional interaction between the two receptors and providing more evidence that AT1R activation increases CXCR2 expression on the plasma membrane. This in turn provides a mechanism contributing to enhanced inflammatory signalling mediated by CXCR2 and therefore a rationale for inhibiting AT1R as well as CXCR2 to reduce inflammation in conditions such as chronic obstructive pulmonary disease.

1. A pharmaceutical formulation comprising:
a) at least one angiotensin type 1 receptor (AT1R) blocker; and
b) at least one CXC chemokine receptor 2 (CXCR2) pathway inhibitor.

2. The pharmaceutical formulation of claim 1 wherein the formulation is for use in the treatment, amelioration or prevention of a condition or disease that is COPD.

3. The pharmaceutical formulation of claim 2 wherein the COPD is selected from: emphysema, chronic bronchitis, bronchiectasis, and refractory (non-reversible) asthma.

4. The pharmaceutical formulation of claim 1 wherein the CXCR2 inhibitor and the AT₁R blocker are administered:
i) in the same dosage form;
ii) in separate dosage forms.

5. The pharmaceutical formulation of claim 1 wherein the CXCR2 inhibitor and the AT₁R blocker are administered:
i) concurrently;
ii) sequentially.

6. The pharmaceutical formulation of claim 1 wherein the CXCR2 inhibitor is selected from: a direct CXCR2 antagonist, negative allosteric CXCR2 modulator, inverse agonist or allosteric inverse agonist.

7. A method for the treatment, amelioration or prevention of a condition or disease, said method comprising the step of:
i) administering to a subject a therapeutically effective amount of a combination of (a) an angiotensin type 1 receptor (AT₁R) blocker and (b) a CXC chemokine receptor 2 (CXCR2) pathway inhibitor.

8. Use of (a) at least one angiotensin type 1 receptor (AT₁R) blocker, and (b) at least one CXC chemokine receptor 2 (CXCR2) pathway inhibitor; for the manufacture of a dosage form for the treatment or prevention of a condition or disease.

9. A kit for the treatment, amelioration or prevention of a condition or disease, said kit comprising:
i) at least one angiotensin type 1 receptor (AT₁R) blocker;
ii) at least one CXC chemokine receptor 2 (CXCR2) pathway inhibitor; and
iii) instructions for use.

10. The method of claim 7, use of claim 8 or kit of claim 9 wherein the formulation is for use in the treatment, amelioration or prevention of a condition or disease that is COPD.

11. The method, use or kit of claim 10 wherein the COPD is selected from: emphysema, chronic bronchitis, bronchiectasis, and refractory (non-reversible) asthma.

12. The method of claim 7, use of claim 8 or kit of claim 9 wherein the CXCR2 inhibitor and the AT₁R blocker are administered:
i) in the same dosage form;
ii) in separate dosage forms.

13. The method of claim 7, use of claim 8 or kit of claim 9 wherein the CXCR2 inhibitor and the AT₁R blocker are administered:
i) concurrently;
ii) sequentially.

14. The method of claim 7, use of claim 8 or kit of claim 9 wherein the CXCR2 inhibitor is selected from: a direct CXCR2 antagonist, negative allosteric CXCR2 modulator, inverse agonist or allosteric inverse agonist.

15. At least one AT₁R blocker, and at least one CXCR2 inhibitor, for use in a formulation for the treatment, amelioration or prevention of a disease.

16. At least one AT₁R blocker for use in a formulation for the treatment, amelioration or prevention of a disease wherein the at least one AT₁R blocker is administered to the subject concurrently or sequentially with at least one CXCR2 inhibitor.

17. At least one CXCR2 inhibitor for use in a formulation for the treatment, amelioration or prevention of a disease wherein the at least one CXCR2 inhibitor is administered to the subject concurrently or sequentially with at least one AT₁R blocker.

18. The at least one AT₁R blocker and the at least one CXCR2 inhibitor for use according to any one of claims 15 to 17 wherein the disease is COPD.

19. The at least one AT₁R blocker and the at least one CXCR2 inhibitor for use according to claim 18 wherein the COPD is a disease selected from: emphysema, chronic bronchitis, bronchiectasis, and refractory (non-reversible) asthma.

20. The at least one AT₁R blocker for use or the at least one CXCR2 inhibitor for use according to any one of claims 15 to 17, wherein the at least one AT₁R blocker and/or at least one CXCR2 inhibitor are administered:
i) in separate dosage forms;
ii) in the same dosage form.

21. The at least one AT₁R blocker and the at least one CXCR2 inhibitor for use according to any one of claims 15 to 17 wherein the at least one AT₁R blocker and the at least one CXCR2 inhibitor are administered:
i) concurrently;
ii) sequentially.

22. The at least one CXCR2 inhibitor for use according to any one of claims 15 to 17 wherein the CXCR2 inhibitor is selected from: a direct CXCR2 antagonist, negative allosteric CXCR2 modulator, inverse agonist or allosteric inverse agonist.

23. The at least one AT₁R blocker and/or at least one CXCR2 inhibitor, for use according to any one of claims 15 to 17, wherein the dosage form containing the AT₁R blocker comprises about 5 mg to 1 g of the AT₁R blocker, and the dosage form containing the CXCR2 inhibitor comprises about 5 mg to 1 g of the CXCR2 inhibitor.
